(19) United States
(12) Patent Application Publication
(10) Pub. No.: US 2023/0341394 A1
(43) Pub. Date: Oct. 26, 2023

(54) TR-FRET BASED ASSAY FOR DETECTION OF ANTIBODIES IN SEROLOGICAL SAMPLES

(71) Applicants: DANA-FARBER CANCER INSTITUTE, INC., Boston, MA (US); THE GENERAL HOSPITAL CORPORATION, Boston, MA (US); PRESIDENT AND FELLOWS OF HARVARD COLLEGE, Cambridge, MA (US)

(72) Inventors: Eric Fischer, Newton, MA (US); Radoslaw Nowak, Boston, MA (US); Hong Yue, Boston, MA (US); Daan Overwijn, Boston, MA (US); Ralph Mazitschek, Weston, MA (US); Neil Connor Payne, Cambridge, MA (US)

(73) Assignees: DANA-FARBER CANCER INSTITUTE, INC., Boston, MA (US); THE GENERAL HOSPITAL CORPORATION, Boston, MA (US); PRESIDENT AND FELLOWS OF HARVARD COLLEGE, Cambridge, MA (US)

(21) Appl. No.: 18/020,800

(22) PCT Filed: Aug. 16, 2021
(86) PCT No.: PCT/US2021/046161

§ 371 Date: Feb. 10, 2023

Related U.S. Application Data
(60) Provisional application No. 63/066,632, filed on Aug. 17, 2020.

(51) Int. Cl. G01N 33/569; G01N 33/542

(52) U.S. Cl. CPC G01N 33/56983; G01N 33/542; G01N 2333/165; G01N 2469/20

(57) ABSTRACT

The present invention relates to a homogeneous, time resolved, Förster resonance energy transfer (TR-FRET)-based method for detection of SARS-CoV-2, SARS CoV-1, and MERS-CoV antibodies in a patient fluid sample.

### RELATED APPLICATIONS

This application claims the benefit of priority under 35 U.S.C. § 119(e) to U.S. Provisional Application No. 63/066,632, filed Aug. 17, 2020, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Aug. 13, 2021, is named 52095_707001WO_ST25.txt and is 56.3 KB bytes in size.

### BACKGROUND OF THE INVENTION

The current pandemic, known as Coronavirus disease 2019, or COVID-19, is caused by acute respiratory syndrome coronavirus 2 (SARS-CoV-2). Effective treatment options are limited. While a vaccine is the ultimate goal, development is at best months away, perhaps even years. Furthermore, it remains to be shown whether long-lasting immunity will be elicited. Therefore, a better understanding of this virus, the disease caused by the virus, the host immune response to the virus, and the prevalence of protective antibodies in naive and in the future vaccinated individuals remain critically important.

Nucleic acid-based detection of viral RNA is the predominantly used method to detect SARS-CoV-2-infected patients. These tests, however, can only detect an infection during the period of active disease, which is relatively short in duration. Robust serological assays have proven advantageous in this respect. They can detect the presence of SARS-CoV-2-specific antibodies and are capable of detecting individuals who have experienced past infection even when asymptomatic. Hence, they have become an important diagnostic tool for managing the current pandemic.

For instance, multiple serological assays for the detection of SARS-CoV-2 antibodies are now being tested in clinical studies. These largely belong to the group of enzyme-linked immunosorbent assays (ELISA). The ELISA assay format has been the gold standard in the diagnostics industry for antibody detection. Still, ELISA has several common limitations, among which include throughput time and costs inherent to the assay format. For example, ELISA tests are relatively slow (approximately 4-6 hours for each run) and require specialized automation. In the case of detecting SARS-CoV-2 antibodies, these limitations have proven quite problematic from the standpoints of widespread use and the number of individuals who can be tested.

### SUMMARY OF THE INVENTION

The present invention includes a rapid mix-and-read assay that may accurately detect seroconversion in patients suffering from a betacoronavirus (β-CoV) infection, e.g., an acute respiratory syndrome coronavirus 1 (SARS-CoV-1), acute respiratory syndrome coronavirus 2 (SARS-CoV-2), or Middle East Respiratory Syndrome-related coronavirus (MERS-CoV) infection, in very small volumes of fluid samples, and with high sensitivity and specificity. The present assay addresses the important need for robust, simple implementation, and scalable serological tests.

The present invention exploits a phenomenon known as Förster resonance energy transfer, also known as fluorescence resonance energy transfer (FRET). FRET is a distance-dependent physical process. When an excited molecular fluorophore (referred to herein as the donor fluorophore) is brought into close proximity (e.g., within 10 nm) with another fluorophore (referred to herein as the acceptor fluorophore), energy is transferred non-radiatively from the donor to the acceptor by means of intermolecular long-range dipole-dipole coupling. Upon excitation at a characteristic wavelength, the energy absorbed by the donor fluorophore is transferred to the acceptor, which in turn emits the energy, referred to herein as the FRET signal. The nature of the signal, and means for detecting or measuring it, are known in the art. As explained in more detail herein, the assays are time-resolved (TR) as well, which provide even greater sensitivity and accuracy. Further, the inventive methods (assays) are homogeneous, which allow for fast reaction times (e.g., taking seconds to minutes), a single incubation of the sample and reagent(s) which may be pre-mixed, and without a solid phase or any washing steps.

Accordingly, one aspect of the present invention provides a homogeneous, TR-FRET-based method for detection of a betacoronavirus (β-CoV) (e.g., SARS-CoV-2) antibodies in a patient fluid sample. The betacoronavirus (β-CoV) (e.g., SARS CoV-2) antibodies that are detected by the present methods are referred to herein as primary antibodies. To test a body fluid sample from a patient, the inventive methods employ two reagents, each of which binds to a betacoronavirus (β-CoV) (e.g., SARS CoV-2) antibody. One of the reagents includes a betacoronavirus (β-CoV) (e.g., SARS CoV-2) antigen. As used herein, a betacoronavirus (β-CoV) antigen refers to any protein or portion thereof capable of eliciting an antibody response in a patient infected with a betacoronavirus (β-CoV) such as SARS-CoV-2. The second reagent may include the same betacoronavirus (β-CoV) antigen. In some embodiments, the second reagent may include a secondary antibody that binds to the primary antibody. In some embodiments, the second reagent may include a nanobody that binds the primary antibody. The reagents are differentially labeled with a donor fluorophore and an acceptor fluorophore. By “differentially” it is meant that the two reagents are labeled with relative concentrations of the fluorophore donor and the fluorophore acceptor sufficient to generate a detectable FRET signal. Therefore, in embodiments wherein the first and second reagents both include a β-CoV antigen, a subpopulation of the β-CoV antigen is labelled with the donor fluorophore and a second subpopulation of the β-CoV antigen is labeled with the acceptor fluorophore.

The body fluid sample is brought into contact with the differentially labeled reagents in a homogeneous assay format, thus forming an assay mixture. Anti-β-CoV antibodies (e.g., anti-SARS-CoV-2 antibodies) present in the fluid sample will bind both of the differentially labeled reagents, bringing the donor and acceptor fluorophores into close proximity. These binding events generate a detectable FRET signal, diagnostic of the presence of anti-β-CoV antibodies (e.g., anti-SARS-CoV-2 antibodies) and infection with the virus. Conversely, the lack of a FRET signal indicates an absence of anti-β-CoV antibodies (e.g., SARS CoV-2 antibodies) and no infection with a β-CoV virus.

The methods offer flexibility and allow for detection of anti-β-CoV antibodies (e.g., anti-SARS-CoV-2 antibodies), per se, as well as specific classes and subclasses of antibodies.

Accordingly, in some embodiments, the methods are designed to detect presence of β-CoV antibodies (e.g., SARS-CoV-2 antibodies) without regard to their class or subclass. A patient fluid sample such as whole blood, plasma or serum is contacted with a β-CoV antigen (e.g., a SARS-CoV-2 antigen) differentially labeled with a donor fluorophore and an acceptor fluorophore. The β-CoV antigen (e.g., SARS-CoV-2 antigen) labeled with the donor fluorophore is considered as the first reagent. The β-CoV antigen (e.g., SARS CoV-2 antigen) labeled with the acceptor fluorophore is considered as the second reagent. The body fluid sample is brought into contact with the differentially labeled reagents in a homogeneous assay format. Due to the multi-valent properties of antibodies in general, if anti-β-CoV antibodies (e.g., SARS-CoV-2 antibodies) are present in the sample, they will be bound by the first and second reagents, bringing the donor and acceptor fluorophores into close proximity, resulting in generation of a FRET signal. Detection of the FRET signal indicates presence of anti-β-CoV antibodies (e.g., anti-SARS-CoV-2 antibodies) in the fluid sample, and hence diagnosis of β-CoV infection (e.g., SARS-CoV-2 infection). These embodiments detect the presence of anti-β-CoV antibodies (e.g., anti-SARS-CoV-2 antibodies). They are not specific as to which class (e.g., IgG, IgM and/or IgA) or subclass (e.g., IgA1, IgA2, etc.) the anti-β-CoV antibodies (e.g., SARS-CoV-2 antibodies) belong.

Accordingly, in other embodiments, the methods assess specific classes or subtypes of anti-β-CoV antibodies (e.g., anti-SARS-CoV-2 antibodies) present in a patient fluid sample. To achieve this additional level of specificity, the reagents are selected such that the first reagent includes a β-CoV antigen (e.g., a SARS-CoV-2 antigen), and the second reagent includes a secondary (e.g., mammalian) antibody that specifically binds to a specific class or subclass of human antibodies. The secondary antibody does not have to be a human antibody. It may originate from any non-human species such as a goat or rodent (e.g., mouse) so long as it specifically detects the human anti-SARS CoV-2 antibodies generated by the patient being tested. In some embodiments, the second reagent includes a nanobody that specifically binds to a specific class or subclass of human antibodies. The two reagents are differentially labeled with the donor and acceptor fluorophores. Therefore, the β-CoV antigen (e.g., SARS-CoV-2 antigen) is labeled with the donor fluorophore, the secondary antibody or nanobody is labeled with the acceptor fluorophore, and vice-versa. The secondary antibody may be a standard anti-IgG, anti-IgM or anti-IgA antibody, for example. Due to the affinity of the secondary antibody or nanobody, if the primary, anti-SARS-CoV-2 antibodies present in the sample include IgG antibodies, for example, they will bind with an anti-IgG secondary antibody or nanobody. The body fluid sample is brought into contact with the differentially labeled reagents in the homogeneous assay format. If β-CoV antibodies (e.g., SARS-CoV-2 antibodies) of a specific class or subclass are present in the sample, they will be bound by the first and second reagents, bringing the donor and acceptor fluorophores into close proximity, resulting in generation of a FRET signal. Detection of the FRET signal indicates presence of anti-β-CoV antibodies (e.g., anti-SARS-CoV-2 antibodies) of a specific class or subclass in the fluid sample, and hence diagnosis of SARS-CoV-2 infection.

Another aspect of the present invention relates to assay reagents, per se. In some embodiments, the pair of reagents includes a β-CoV antigen (e.g., a SARS-CoV-2 antigen) differentially labeled with a fluorophore donor and a fluorophore acceptor.

In some embodiments, the pair of reagents includes as the first reagent, a β-CoV antigen (e.g., a SARS-CoV-2 antigen) labeled with a fluorophore donor or a fluorophore acceptor. The second reagent is an anti-anti-β-CoV antibody (e.g., an anti-anti-SARS-CoV-2 antibody) or a nanobody labeled with a fluorophore donor or a fluorophore acceptor, provided that the fluorophore donor and acceptor are disposed on different reagents. In some embodiments, second reagent is an anti-IgG antibody or an anti-IgG nanobody. In some embodiments, the second reagent is an anti-IgM antibody or an anti-IgM nanobody. In some embodiments, the second reagent is an anti-IgA antibody or an anti-IgA nanobody, or any subtype thereof, e.g., anti-IgA1, IgA2.

A further aspect of the present invention is directed to an assay kit for homogeneous, TR-FRET-based method for detection of anti-β-CoV antibodies (e.g., SARS-CoV-2 antibodies) in a patient fluid sample, comprising: a) first and second reagents comprising a first subpopulation of a β-CoV antigen (e.g., a SARS-CoV-2 antigen) and a second subpopulation of the β-CoV antigen, respectively, wherein the first and second subpopulations are differentially labeled with a donor fluorophore and an acceptor fluorophore, wherein the first and second reagents may be disposed in the same or different containers; orb) a first reagent comprising a β-CoV antigen (e.g., a SARS-CoV-2 antigen) and a second reagent comprising at least one secondary antibody or a nanobody that binds the anti-β-CoV antibody, wherein the first and second reagents are differentially labeled with the donor fluorophore and the acceptor fluorophore, wherein the at least one secondary antibody or the nanobody may bind a specific class or subtype of human antibodies; and wherein the first and second reagents differentially labeled with the fluorophore donor and acceptor fluorophore are disposed in separate containers; and c) printed instructions for using the reagents in the homogeneous, TR-FRET-based method for detection of anti-β-CoV antibodies (e.g., SARS-CoV-2 antibodies) in a patient fluid sample.

In some embodiments of the disclosed methods, assay reagents and kits, the β-CoV antigen is a β-CoV full-length Spike protein, or an antigenic portion thereof (e.g., a full-length SARS CoV-2 Spike protein). In some embodiments, the antigenic portion of the Spike protein is the S1 subunit or the S2 subunit. In some embodiments, the antigenic portion of the Spike protein is the S-receptor binding domain (S-RBD). In some embodiments, the β-CoV antigen is a β-CoV nucleocapsid protein (“N-protein”) or an antigenic fragment thereof (i.e., that binds an anti-β-CoV antibody).

The inventive methods offer significant advantages over the ELISA methods. The present TR-FRET assay can generate results in a fraction of the time required by an ELISA, such as in a 30-45-minute timeframe. The methods do not require complex equipment; they can be performed with a multichannel pipette and a TR-compatible plate reader. Due to its relative simplicity (mix and read), it can be easily implemented even in remote, poorly developed regions. And it can be scaled to hundreds or thousands of tests per day at competitively low costs per sample. The assays are amenable to high-throughput screening, which as known in the art, means that a relatively large number of samples can be analyzed simultaneously, e.g., in multi-well microtiter plates, e.g., in a 96 well plate or a 384-well plate or a plate with 1536 or 3456 wells.

These advantages are magnified in terms of specificity and sensitivity. As demonstrated in the working examples, the assays were evaluated on a test set of 45 polymerase chain reaction (PCR)-positive, and 30 PCR-negative samples that were previously profiled using two different ELISA formats. One format utilized an S-receptor-binding domain (S1-RBD) as the labeled antigen and the other utilized the full S protein as the labeled antigen. Using either 2 or 4 standard deviations as cut-off, comparable or superior performance was observed as compared to both ELISA formats

### BRIEF DESCRIPTION OF THE DRAWINGS

FIG. 1 is a schematic of a SARS-CoV-2 virion particle.

FIG. 2 is a schematic showing the assay setup for TR-FRET serological assay. 1. S-RBD is labeled with BODIPY and Tb at 50:50 ratio and mixed with serum, which will lead to detection of S-RBD specific antibodies but no discrimination between isotypes. 2. Antibodies recognizing human Immunoglobulin G/Immunoglobulin M/Immunoglobulin A1 (IgG/IgM/IgA1) are labeled with Tb. And S-RBD or S are labeled with BODIPY and both mixed with serum for isotype specific antibody detection. 3. As in 2) but with swap of labels. The commonly used ELISA is shown as reference on the right.

FIG. 3 is a line graph showing the dimerization of SARS-CoV-2 RBD domain of Spike protein to detect SARS-CoV-2 specific IgA1, IgA2, IgM, and IgG antibodies.

FIG. 4 is a line graph showing comparison of antibody detection for assay 1, assay 2 and assay 3, and demonstrating differences in assay performance.

FIG. 5A-5C is a schematic and series of scatterplots showing the assay setup and CR3022 validation. FIG. 5A is a schematic showing the scientific mechanism of the TR-FRET assay. Antibodies recognizing human IgG are labeled with BODIPY and SARS-Cov-2 Spike protein are labeled with Tb (Terbium). Both are mixed with serum for isotype specific antibody detection. The light pulse at 337 nm excites Terbium chelate of Tb—S labeled protein and produces light at 490 nm. The energy emitted from chelate is transferred to BODIPY-labeled secondary antibodies in presence of positive primary antibody, generating a TR-FRET signal at 520 nm. FIG. 5B is a flow chart of the TR-FRET assay. The serum samples are diluted into multiple well plates and added into reaction mixture. Reaction mixture is added beforehand by automated dispenser (Multidrop Combi Reagent Dispenser). The diluted serum samples are added into the reaction mixture with a Crystal Gryphon mechanical dispenser (Art Robbins Instruments, LLC) or a manual multichannel pipette. The plate is read on TR-FRET compatible plate reader (PHERAstar FSX Microplate Reader). FIG. 5C is a series of scatterplots showing the titration of CR3022 IgG/IgM/IgA1 into a pre-formed mix of Tb-S protein (7.5 nM final) and BODIPY labeled α-hsIgG/IgM/IgA (250 nM final). FIG. 5D is the same as in FIG. 5C, but in presence of 1:150 dilution of negative serum. Data in FIG. 5B and FIG. 5C are represented as means±s.d of two replicates (n=2).

FIG. 6A-6D is a series of graphs showing optimization of a serum dilution factor. FIG. 6A is a graph showing the sensitivity and specificity of the ELISA IgG assay. Serum samples from a cohort of 49 PCR positive and 28 PCR negative samples (96w_testset) were diluted at 1:100 serum to buffer ratios and processed in ELISA assay. FIG. 6B is a graph showing the sensitivity and specificity for TR-FRET αIgG-S assay on the same cohort at 1:100 dilution. FIG. 6C is a series of graphs showing the correlation of TR-FRET αIgG-S assay using serum dilutions of 1:50, 1:100 or 1:150 to the ELISA αIgG —S assay at 1:100 serum dilution. FIG. 6D is a series of graphs showing the sensitivity and specificity analysis for the TR-FRET αIgG-S assay with the 1:50 and 1:150 dilutions. All the data in FIG. 6A-6D are represented as means±s.d. of three replicates (n=3).

FIG. 7A-7C is a series of graphs showing the sensitivity and specificity of the TR-FRET αIgG-S assay. FIG. 7A is a graph showing the sensitivity and specificity of TR-FRET αIgG-S assay performed on a cohort of 68 SARS-CoV-2 PCR positive samples (CoV2+), and 100 pre-pandemic negative samples (healthy). FIG. 7B is a graph showing the sensitivity and specificity of the ELISA IgG performed on the same cohort. FIG. 7C is a graph showing the correlation of the TR-FRET IgG and ELISA IgG when performed with the same concentration of serum. Data of FIG. 7A-7C are represented as means±s.d of two replicates (n=2).

FIG. 8A-8B is a series of graphs showing the limitation of detection and quantitation. FIG. 8A is a series of graphs showing the results for the TR-FRET αIgG-S assay. Titration of CR3022 IgG was performed in presence and absence of negative serum at 1:100 dilution. Data are represented as means±s.d of three replicates (n=3). The concentration of CR3022 selected for LoD study is highlighted with red arrow. FIG. 8B is a series of graphs showing the limit of detection for the TR-FRET αIgG-S assay, as assessed by comparing 20 replicates (n=20) of the CR3022 to buffer control in the presence and absence of negative serum at 1:100 dilution.

FIG. 9A-9B is a series of graphs showing that FIG. 8 TR-FRET is compatible with other antigens. FIG. 9A is a graph showing the sensitivity and specificity of the TR-FRET αIgG-N assay performed on a cohort of 45 PCR positive and 30 PCR negative samples (96w_testset). FIG. 9B is a graph showing the correlation of the TR-FRET αIgG-S assay to the TR-FRET αIgG-N assay performed on the same 96w_testset. Data of FIG. 9A-9B are represented as means±s.d of two replicates (n=2).

FIG. 10A-10G is a series of graphs and schematics. FIG. 10A is a graph showing the titration of BODIPY labeled CR3022 IgG antibody into Tb-labeled RBD mix (15 nM final concentration). The Binding affinity of CR3022 and RBD (Kd app) was calculated using a nonlinear regression fit model (GraphPad Prism Software). Data are represented as means±s.d of three replicates (n=3). FIG. 10B is a schematic showing alternative labelling strategies for the TR-FRET assay. Donor fluorophore is located on the antigen (RBD) and the acceptor fluorophore on the detection antibody (αIgG/M/A). FIG. 10C is schematic showing alternative labelling strategies for the TR-FRET assay. Donor fluorophore is located on the detection antibody (αIgG/M/A) and the acceptor fluorophore is located on the antigen (RBD). FIG. 10D is a series of graphs showing the titration of CR3022 IgG/IgM/IgA1 in the same assay setup as in FIG. 10B. FIG. 10E is a graph showing the titration of CR3022 IgG/IgM/IgA1 in the same assay setup as in FIG. 10C. FIG. 10F is a graph showing the titration of CR3022 IgG/IgM/IgA1 in presence of 1:150 negative serum, assay setup as in FIG. 10B. FIG. 10G is a graph showing the titration of CR3022 IgG/IgM/IgA1 in the presence of 1:150 negative serum in the same assay setup as in FIG. 10C. Data of FIG. 10C-10F are represented as means±s.d of two replicates (n=2).

FIG. 11A-11C is a series of graphs showing the optimization of antigen amount, detection of antibody amount, and detection of antibody dilutions. FIG. 11A is a graph showing the titration of CR3022 IgG into BODIPY-αIgG (250 nM final concentration) with varying concentrations of Tb—S protein. FIG. 11B is a graph showing the titration of CR3022 IgG into Tb—S (7.5 nM final concentration) with varying concentrations of BODIPY-αIgG. FIG. 11C is a graph showing the titration of positive and negative serum in final assay conditions of 250 nM BODIPY-αIgG and 7.5 nM Tb—S. Data in FIG. 11A-11C is singlicate (n=1).

FIG. 12A-12B is a series of graphs showing the optimization of the degree of labelling for Tb—S protein. FIG. 12A is a graph showing the titration of CR3022 IgG into BODIPY-αIgG (250 nM final concentration) with Tb—S (7.5 nM final concentration) for varying degrees of labelling. FIG. 12B is a graph showing the titration of positive or negative serum into BODIPY-αIgG (250 nM final concentration) with Tb—S (7.5 nM final concentration) for varying degrees of labelling. Data in FIG. 12A-12B is singlicate (n=1).

FIG. 13A-13B is a series of graphs and a table showing assay precision. FIG. 13A is a series of graphs showing comparison among three independent TR-FRET αIgG-S assays performed on different days by three different operators on a set of positive responder and negative control samples (68 total). FIG. 13B is a table showing the calculated average assay repeatability across operators (CV %) and average intermediate precision (calculated across days and operators), with results corresponding to data in FIG. 8A. Data of FIG. 13C-13F are represented as means±s.d of two replicates (n=2).

FIG. 14 is a graph showing the results of a TR-FRET IgG-N Assay. Titration of positive and negative serum was performed into BODIPY-αIgG (250 nM final concentration) with biotinylated N (10 nM final concentration) and Tb-Streptavidin (4 nM final concentration). Data in FIG. 14 is singlicate (n=1).

FIG. 15 is a graph of S-IgG ELISA response vs. S-IgG TR-FRET response showing the correlation of the TR-FRET and ELISA assays.

FIG. 16 is a set of graphs comparing S-IgG response of serum samples and self-collection samples (Neoteryx) obtained from the same subjects, measured by ELISA and TR-FRET.

FIG. 17 is an exemplary set of SNR and Z′ calculations comparing S-IgG ELISA and S-IgG TR-FRET against the CoV2− set, extracted from the data set in FIG. 16.

FIG. 18 is a graph of TR-FRET ratio versus concentration for a titration of CR3022 positive control antibody or CR3022 in the presence of 1:150 SARS-CoV2 negative serum (CoV2−), or CoV2+or CoV2− serum sample into 7.5 nM Tb—S, 250 nM AF488-Anti-IgG-Nanobody, showing that replacing BODIPY-anti-IgG antibody with AF488-anti-IgG-Nanobody can successfully detect CR3022 antibody binding to S protein with or without negative control serum. Furthermore, this setup is capable of detection of positive and negative control serum.

FIG. 19 is a graph of TR-FRET ratio versus concentration for a titration of CR3022 positive control antibody or CR3022 in presence of 1:150 SARS-CoV2 negative serum (CoV2−), or CoV2+ or CoV2− serum sample into 7.5 nM Tb-anti-IgG-Nanobody, 250 nM BODIPY-S, showing that replacing Tb-anti-IgG antibody with Tb-Nanobody can successfully detect CR3022 antibody binding to S protein with or without negative control serum. Furthermore, this setup is capable of detection of positive and negative control serum.

FIG. 20 is a graph of TR-FRET ratio versus concentration for a titration of CR3022 positive control antibody or CR3022 in presence of 1:150 SARS-CoV2 negative serum (CoV2−), or CoV2+ or CoV2− serum sample into 7.5 nM Tb-anti-IgG-Nanobody, 250 nM BODIPY-S with y-axis scale between 0 and 1.

FIG. 21 is a graph of TR-FRET ratio versus concentration showing a comparison between Tb—S and AF488-anti-IgG Nanobody, Tb—S and BODIPY-anti-IgG antibody and Tb-anti-IgG Nanobody and BODIPY-S, showing that replacing Anti-IgG antibody with an Anti-IgG nanobody was able to successfully detect control CR3022 antibody in all cases

FIG. 22 is a graph of TR-FRET ratio versus concentration showing a comparison between Tb—S and AF488-anti-IgG Nanobody, Tb—S and BODIPY-anti-IgG antibody and Tb-anti-IgG Nanobody and BODIPY-S all in the presence of 1:150 dilution of SARS-CoV2 negative serum, showing that all methods were able to successfully detect CR3022 in presence of negative control serum. Replacing BODIPY-anti-IgG antibody with AF488-anti-IgG Nanobody improved assay signal more than two-fold (from 0.25 to 0.6) in the presence of 1:150 dilution of SARS-CoV2 negative serum.

FIG. 23 is a graph of TR-FRET ratio versus concentration for a titration of SARS-CoV2 positive or negative serum in 7.5 nM Tb—S, 250 nM AF488-anti IgG Nanobody final concentrations, showing that fluorescent AF488-anti IgG Nanobody used instead of fluorescently labelled anti IgG antibody was able to detect and discriminate between SARS-CoV2 positive and negative serum.

FIG. 24 is a graph of TR-FRET ratio versus concentration for a titration of SARS-CoV2 positive or negative serum in 7.5 nM Tb-anti IgG Nanobody, 250 nM BODIPY-S final concentrations, showing that replacing Tb-anti IgG antibody with Tb-anti IgG-Nanobody can detect and discriminate between SARS-CoV2 positive and negative samples.

FIG. 25A-FIG. 25I is a series of graphs that show that TR-FRET is compatible with other antigens. FIG. 25A shows the sensitivity and specificity of TR-FRET αIgG-S protein assay performed on MassCPR set including 90 pre-pandemic negative samples and 100 SARS-CoV-2 positive. FIG. 25B shows the sensitivity and specificity of TR-FRET αIgG-N protein assay performed on MassCPR. FIG. 25C shows the correlation of αIgG-S titer in TR-FRET assay versus ELISA assay for MassCPR. Note the ‘ceiling’ of signal in ELISA assay and high dynamic range of TR-FRET. FIG. 25D shows the correlation of IgG titer N protein in TR-FRET assay versus ELISA assay for MassCPR. FIG. 25E shows the hospital admission status of the 100 SARS-CoV-2 positive cohort. ER—Emergency Room, IP—inpatient, OP—outpatient. FIG. 25F shows the IgG titer as measured by TR-FRET assay stratified by number of days since last positive SARS-CoV-2 test. FIG. 25G shows the correlation of TR-FRET αIgG-S and TR-FRET αIgG-N assays performed on MassCPR indicates diverse immune response to different antigens. FIG. 25H shows the cross reactivity between S proteins of SARS-CoV-2 and SARS-CoV measured by TR-FRET IgG titer on MassCPR. FIG. 25I, as in FIG. 25H, shows the cross-reactivity, but for S proteins of SARS-CoV-2 and MERS-CoV. Data are represented as means±SD of two replicates (n=2).

FIG. 26A-FIG. 26E is a series of graphs that show the optimization of degree of labeling of Tb—S protein and TR-FRET αIgG-N protein assay. FIG. 26A shows the titration of positive and negative serum into BODIPY-αIgG Ab (250 nM final) with biotinylated N (20 nM final) and Tb-SA (2 nM final). Data is singlicate (n=1). FIG. 26B shows the sensitivity and specificity of TR-FRET IgG-N assay performed on 96w_testset using N protein. Data is shown as duplicates (n=2). FIG. 26C shows the correlation of TR-FRET αIgG-S to TR-FRET αIgG-N assays performed on the 96w_testset. Data are represented as means±SD of two replicates (n=2). FIG. 26D shows the sensitivity and specificity of ELISA IgG-S assay performed on MassCPR (90 pre-pandemic negative samples and 100 SARS-CoV-2 positive). Data represented as means±SD of four replicates (n=4). FIG. 26E shows the sensitivity and specificity of ELISA IgG-N assay performed on MassCPR. Data represented as means±SD of four replicates (n=4).

### DETAILED DESCRIPTION OF THE INVENTION

### Reagents

The disclosed methods employ two labeled reagents, at least one of which is a β-CoV antigen (e.g., a SARS-CoV-2 antigen) that is labeled with a donor fluorophore or an acceptor fluorophore. As used herein, a β-CoV antigen refers to any protein or portion thereof of a β-CoV virion that is capable of eliciting an antibody response in a patient infected with a β-CoV.

### First Reagent

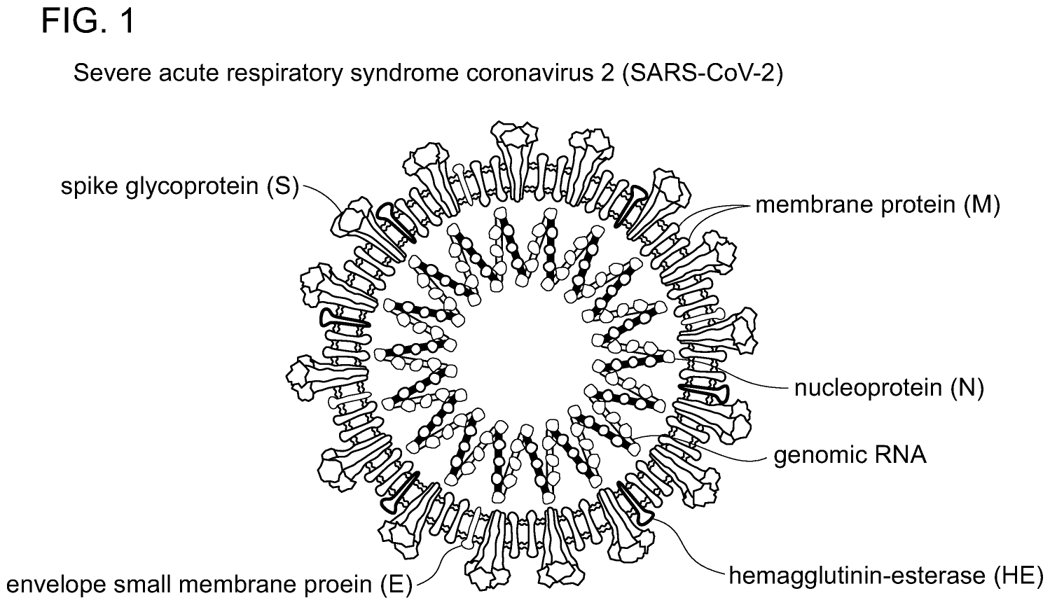

A schematic of a SARS-CoV/MERS virion particle is illustrated in FIG. 1. As shown therein, the so-called spike proteins are the visible protrusions on the surface of a β-CoV virion, giving these viruses their characteristic, crown-like appearance. These homotrimeric proteins are heavily glycosylated, with each comprising two distinct subunits: S1 and S2. The role of Spike is to act as a molecular key, achieved by recognizing and binding to specific ACE2 cell-surface receptors (the locks) present on the surface of human cells, via the S1 receptor-binding domain. When S1-RBD binds to acetylcholinesterase 2 (ACE2), Spike undergoes dramatic structural changes to alter the conformation of ACE2 and mediate entry of the virus into the host cell.

Spike proteins, projecting into the external environment and effectively binding cell-surface receptors, become exposed to recognition by the immune system. This makes Spike the immunodominant coronavirus antigen, causing it to elicit a strong neutralizing antibody response. (Ju, et al., Nature 584, 115-19 (2020)).

Accordingly, in some embodiments, the inventive methods and reagents employ a full-length Spike protein of a β-CoV, or an antigenic portion thereof. In some embodiments, the antigenic portion of the Spike protein is the S1 subunit or the S2 subunit. In some embodiments, the antigenic portion of the Spike protein is the S1-receptor binding domain (S1-RBD).

An exemplary amino acid sequence of the Spike protein of a SARS CoV-2 virus [Human coronavirus NL63] is provided at NCBI Accession No. YP_003767, version YP_003767.1, incorporated herein by reference, and reproduced below (SEQ ID NO: 1):

| | |
|---|---|
| 1 | mklflillvl plascfftcn snanlsmlql gvpdnsstiv tgllpthwfc anqstsvysa |
| 61 | ngffyidvgn hrsafalhtg yydanqyyiy vtneiglnas vtlkickfsr nttfdflsna |
| 121 | sssfdcivnl lfteqlgapl gitisgetvr lhlynvtrtf yvpaaykltk lsvkcyfnys |
| 181 | cvfsvvnatv tvnvtthngr vvnytvcddc ngytdnifsv qqdgripngf pfnnwflltn |
| 241 | gstlvdgvsr lyqplrltcl wpvpglksst gfvyfnatgs dvncngyqhn svvdvmrynl |
| 301 | nfsansldnl ksgvivfktl qydvlfycsn sssgvldtti pfgpssqpyy cfinstintt |
| 361 | hvstfvgilp ptvreivvar tgqfyingfk yfdlgfieav nfnvttasat dfwtvafatf |
| 421 | vdvlvnvsat niqnllycds pfeklqcehl qfglqdgfys anflddnvlp etyvalpiyy |
| 481 | qhtdinftat asfggscyvc kphqvnisln gntsvcvrts hfsiryiynr vksgspgdss |
| 541 | whiylksgtc pfsfsklnnf qkfkticfst vevpgscnfp leatwhytsy tivgalyvtw |
| 601 | segnsitgvp ypvsgirefs nlvlnnctky niydyvgtgi irssnqslag gityvsnsgn |
| 661 | llgfknvstg nifivtpcnq pdqvavyqqs iigamtavne sryglqnllq lpnfyyvsng |
| 721 | gnncttavmt ysnfgicadg slipvrprns sdngisaiit anlsipsnwt tsvqveylqi |
| 781 | tstpivvdca tyvengnprc knllkqytsa cktiedalrl sahletndvs smltfdsnaf |
| 841 | slanvtsfgd ynlssvlpqr nirssriagr saledllfsk vvtsglgtvd vdyksctkgl |
| 901 | siadlacaqy yngimvlpgv adaermamyt gsliggmvlg gltsaaaipf slalqarlny |
| 961 | valqtdvlqe nqkilaasfn kainnivasf ssvndaitqt aeaihtvtia lnkiqdvvnq |
| 1021 | qgsalnhlts qlrhnfqais nsiqaiydrl dsiqadqqvd rlitgrlaal nafvsqvlnk |
| 1081 | ytevrgsrrl aqqkinecvk sqsnrygfcg ngthifsivn sapdgllflh tvllptdykn |
| 1141 | vkawsgicvd giygyvlrqp nlvlysdngv frvtsrvmfq prlpvlsdfv qiyncnvtfv |
| 1201 | nisrvelhtv ipdyvdvnkt lqefaqnlpk yvkpnfdltp fnltylnlss elkqleakta |
| 1261 | slfqttvelq glidqinsty vdlkllnrfe nyikwpwwvw liisvvfvvl lsllvfccls |
| 1321 | tgccgccncl tssmrgccdc gstklpyyef ekvhvq |

The S1 subunit is located between amino acid residues 17 to 680. The S2 subunit is located between residues 727 to 1195. The S1-RBD is located at residues 331 to 524 of the S protein.

Another exemplary SARS-CoV-2 spike amino acid sequence, provided at UniProtKB-PODTC2, is herein incorporated by reference and is reproduced below (SEQ ID NO: 2):

| | |
|---|---|
| MFVFLVLLPL VSSQCVNLTT RTQLPPAYTN SFTRGVYYPD KVFRSSVLHS TQDLFLPFFS | 60 |
| NVTWFHAIHV SGTNGTKRFD NPVLPFNDGV YFASTEKSNI IRGWIFGTTL DSKTQSLLIV | 120 |
| NNATNVVIKV CEFQFCNDPF LGVYYHKNNK SWMESEFRVY SSANNCTFEY VSQPFLMDLE | 180 |
| GKQGNFKNLR EFVFKNIDGY FKIYSKHTPI NLVRDLPQGF SALEPLVDLP IGINITRFQT | 240 |
| LLALHRSYLT PGDSSSGWTA GAAAYYVGYL QPRTFLLKYN ENGTITDAVD CALDPLSETK | 300 |
| CTLKSFTVEK GIYQTSNERV QPTESIVRFP NITNLCPFGE VFNATRFASV YAWNRKRISN | 360 |
| CVADYSVLYN SASFSTFKCY GVSPTKLNDL CFTNVYADSF VIRGDEVRQI APGQTGKIAD | 420 |
| YNYKLPDDFT GCVIAWNSNN LDSKVGGNYN YLYRLFRKSN LKPFERDIST EIYQAGSTPC | 480 |
| NGVEGFNCYF PLQSYGFQPT NGVGYQPYRV VVLSFELLHA PATVCGPKKS TNLVKNKCVN | 540 |
| FNFNGLTGTG VLTESNKKFL PFQQFGRDIA DTTDAVRDPQ TLEILDITPC SFGGVSVITP | 600 |
| GTNTSNQVAV LYQDVNCTEV PVAIHADQLT PTWRVYSTGS NVFQTRAGCL IGAEHVNNSY | 660 |
| ECDIPIGAGI CASYQTQTNS PRRARSVASQ SIIAYTMSLG AENSVAYSNN SIAIPTNFTI | 720 |
| SVTTEILPVS MTKTSVDCTM YICGDSTECS NLLLQYGSFC TQLNRALTGI AVEQDKNTQE | 780 |
| VFAQVKQIYK TPPIKDFGGF NFSQILPDPS KPSKRSFIED LLFNKVTLAD AGFIKQYGDC | 840 |
| LGDIAARDLI CAQKFNGLTV LPPLLTDEMI AQYTSALLAG TITSGWTFGA GAALQIPFAM | 900 |
| QMAYRFNGIG VTQNVLYENQ KLIANQFNSA IGKIQDSLSS TASALGKLQD VVNQNAQALN | 960 |
| TLVKQLSSNF GAISSVLNDI LSRLDKVEAE VQIDRLITGR LQSLQTYVTQ QLIRAAEIRA | 1020 |
| SANLAATKMS ECVLGQSKRV DFCGKGYHLM SFPQSAPHGV VFLHVTYVPA QEKNFTTAPA | 1080 |
| ICHDGKAHFP REGVFVSNGT HWFVTQRNFY EPQIITTDNT FVSGNCDVVI GIVNNTVYDP | 1140 |
| LQPELDSFKE ELDKYFKNHT SPDVDLGDIS GINASVVNIQ KEIDRLNEVA KNLNESLIDL | 1200 |
| QELGKYEQYI KWPWYIWLGF IAGLIAIVMV TIMLCCMTSC CSCLKGCCSC GSCCKFDEDD | 1260 |
| SEPVLKGVKL HYT | 1273 |

The S1-RBD is located at residues 318 to 541.

Yet other SARS-CoV-2 Spike proteins that may be useful reagents in the practice of the present assay methods are known in the art (e.g., available from the NCBI virus database, accession numbers QMT50797, QMT51409, QMT51505, QMT51865, QMT52129, QMT52237, QMT522 49, QMT52393, QMT52561, QMT52741, QMT52765, QMT53017, QMT53041, QMT53053, Q MT53065, QMT53089, QMT53101, QMT53149, QMT53173, QMT53197, QMT53221, QMTS 3233, QMT53245, QMT55880, QMT57260, QMT57332, QMT57572, QMT57584, QMT57608, QMT57644, QMT57656, QMT57692, QMT94108, QMT94756, QMT94780, QMT95200, QM T95308, QMT95356, QMT95368, QMT95452, QMT95488, QMT95560), five from Asia (QLL26046, QLI49781, QLF98260, QKY60061, and QKV26077). SARS CoV Spike proteins and their respective receptor binding domains are also commercially available.

It has been reported that nearly one-third of the spike protein sequence is associated with mutations. Accordingly, mutated versions of the Spike protein (and antigenic, ACE2-binding fragments thereof) may be useful as reagents in the practice of the present assay methods. Mutation sites and mutation types observed in human SARS-CoV-2 spike proteins according to geographical locations are set forth in Table 3 in Guruprasad, Lalitha. “Human SARS CoV-2 spike protein mutations.” Proteins vol. 89, 5 (2021): 569-576. doi:10.1002/prot.26042. Guruprasad found Spike proteins having from 1 to 16 mutations. Referring to SEQ ID NO: 2, Spike proteins having a mutation at any one or more of residues D614, L5, L54, P1263, P681, K417, S477, T859, S221, V483, E484, N501 and A845 may be useful as reagents. Therefore, Spike proteins useful as reagents may have one or more mutations including mutation(s) in the S1-RBD. By way of representative example, in some embodiments, a Spike protein having the mutation D614G (referring to SEQ ID NO: 2) may be used as a reagent. In some embodiments, a Spike protein having the mutation N501Y mutation (referring to SEQ ID NO: 2) may be used as a reagent. In some embodiments, a Spike protein having the mutations K417N, E484K, N501Y (referring to SEQ ID NO: 2) may be used as a reagent. In some embodiments, a Spike protein having the RBD mutations K417T, E484K, and N501Y (referring to SEQ ID NO: 2) may be used as a reagent. In some embodiments, a Spike protein having a mutation in the 18, 69-70, 80, 144, 215, 246, 417, 484, 601, 570, 614, 681, 701, 716, 982, and/or 1118 amino acid position (referring to SEQ ID NO: 2) may be used as reagent.

Yet another exemplary SARS-CoV-2 spike protein amino acid sequence, provided at UniProtKB-P59594, is herein incorporated by reference and is produced below (SEQ ID NO: 3):

| | |
|---|---|
| MFIFLLFLTL TSGSDLDRCT TFDDVQAPNY TQHTSSMRGV YYPDEIFRSD TLYLTQDLFL | 60 |
| PFYSNVTGFH TINHTFGNPV IPFKDGIYFA ATEKSNVVRG WVFGSTMNNK SQSVIIINNS | 120 |
| TNVVIRACNF ELCDNPFFAV SKPMGTQTHT MIFDNAFNCT FEYISDAFSL DVSEKSGNFK | 180 |
| HLREFVFKNK DGFLYVYKGY QPIDVVRDLP SGFNTLKPIF KLPLGINITN FRAILTAFSP | 240 |
| AQDIWGTSAA AYFVGYLKPT TFMLKYDENG TITDAVDCSQ NPLAELKCSV KSFEIDKGIY | 300 |
| QTSNFRVVPS GDVVRFPNIT NLCPFGEVEN ATKFPSVYAW ERKKISNCVA DYSVLYNSTF | 360 |
| FSTFKCYGVS ATKLNDLCFS NVYADSFVVK GDDVRQIAPG QTGVIADYNY KLPDDFMGCV | 420 |
| LAWNTRNIDA TSTGNYNYKY RYLRHGKLRP FERDISNVPF SPDGKPCTPP ALNCYWPLND | 480 |
| YGFYTTTGIG YQPYRVVVLS FELLNAPATV CGPKLSTDLI KNQCVNFNFN GLTGTGVLTP | 540 |
| SSKRFQPFQQ FGRDVSDFTD SVRDPKTSEI LDISPCSFGG VSVITPGTNA SSEVAVLYQD | 600 |
| VNCTDVSTAI HADQLTPAWR IYSTGNNVFQ TQAGCLIGAE HVDTSYECDI PIGAGICASY | 660 |
| HTVSLLRSTS QKSIVAYTMS LGADSSIAYS NNTIAIPTNF SISITTEVMP VSMAKTSVDC | 720 |
| NMYICGDSTE CANLLLQYGS FCTQLNRALS GIAAEQDRNT REVFAQVKQM YKTPTLKYFG | 780 |
| GFNFSQILPD PLKPTKRSFI EDLLFNKVTL ADAGFMKQYG ECLGDINARD LICAQKFNGL | 840 |
| TVLPPLLTDD MIAAYTAALV SGTATAGWTF GAGAALQIPF AMQMAYRENG IGVTQNVLYE | 900 |
| NQKQIANQFN KAISQIQESL TTTSTALGKL QDVVNQNAQA LNTLVKQLSS NFGAISSVLN | 960 |
| DILSRLDKVE AEVQIDRLIT GRLQSLQTYV TQQLIRAAEI RASANLAATK MSECVLGQSK | 1020 |
| RVDFCGKGYH LMSFPQAAPH GVVFLHVTYV PSQERNFTTA PAICHEGKAY FPREGVFVFN | 1080 |
| GTSWFITQRN FFSPQIITTD NTFVSGNCDV VIGIINNTVY DPLQPELDSF KEELDKYFKN | 1140 |
| HTSPDVDLGD ISGINASVVN IQKEIDRLNE VAKNLNESLI DLQELGKYEQ YIKWPWYVWL | 1200 |
| GFIAGLIAIV MVTILLCCMT SCCSCLKGAC SCGSCCKFDE DDSEPVLKGV KLHYT | 1255 |

The S1-RBD is located at residues 318 to 510.

In some embodiments, a Spike protein having a mutation in the 49, 77, 78, 118, 139, 144, 147, 193, 227, 239, 244, 261, 311, 344, 360, 426, 437, 472, 480, 487, 501, 577, 605, 607, 608, 609, 613, 665, 701, 743, 754, 804, 860-861, 894, 999, 1001, 1132, 1148, and/or 1163 amino acid position (referring to SEQ ID NO: 3) may be used as reagent.

As in the case of SARS-CoV-2 full-length Spike proteins, mutated versions of S1-RBD fragments may also be used. Forty-four (44) distinct mutation sites in the S1-RBD have been reported; the mutations are located at positions 337, 344, 345, 348, 354, 357, 367, 368, 379, 382, 384, 393, 395, 403, 407, 408, 411, 413, 441, 453, 457, 458, 468, 471, 476, 477, 479, 483, 484, 485, 486, 491, 493, 494, 498, 500, 501, 506, 507, 508, 518, 519, 520, and 522 (all referring to SEQ ID NO: 2). See, Guruprasad, supra. In some embodiments, an S1-RBD fragment has a mutation at any one of positions 344 (e.g., A344S), 477 (e.g., S477N), 483 (e.g., V483A) and 501 (e.g., N501Y). In some embodiments, an S1-RBD fragment has any one of the following mutations: S477N, V483A, A344S, and N501Y/T. In some embodiments, an S1-RBD fragment has any one of the following mutations: K417N/T, E484K, and N501Y. In some embodiments, an S1-RBD fragment has a mutation at any one of positions Y453 (e.g., Y453F), G476 (e.g., G4765), F486 (e.g., F486L), and T500 (e.g., T5001).

In some embodiments, the first reagent includes a full-length MERS-CoV Spike protein, or a fragment thereof that binds an anti-β-CoV antibody. An exemplary SARS-CoV-1 spike protein amino acid sequence, provided at UniProtKB-R9uQ53, is herein incorporated by reference and produced below (SEQ ID NO: 4):

| | |
|---|---|
| MIHSVFLLMF LLTPTESYVD VGPDSVKSAC IEVDIQQTFF DKTWPRPIDV SKADGIIYPQ | 60 |
| GRTYSNITIT YQGLFPYQGD HGDMYVYSAG HATGTTPQKL FVANYSQDVK QFANGFVVRI | 120 |
| GAAANSTGTV IISPSTSATI RKIYPAFMLG SSVGNFSDGK MGRFFNHTLV LLPDGCGTLL | 180 |
| RAFYCILEPR SGNHCPAGNS YTSFATYHTP ATDCSDGNYN RNASLNSFKE YFNLRNCTFM | 240 |
| YTYNITEDEI LEWFGITQTA QGVHLFSSRY VDLYGGNMFQ FATLPVYDTI KYYSIIPHSI | 300 |
| RSIQSDRKAW AAFYVYKLQP LTFLLDFSVD GYIRRAIDCG FNDLSQLHCS YESFDVESGV | 360 |
| YSVSSFEAKP SGSVVEQAEG VECDFSPLLS GTPPQVYNFK RLVFTNCNYN LTKLLSLFSV | 420 |
| NDFTCSQISP AAIASNCYSS LILDYFSYPL SMKSDLSVSS AGPISQFNYK QSFSNPTCLI | 480 |
| LATVPHNLTT ITKPLKYSYI NKCSRLLSDD RTEVPQLVNA NQYSPCVSIV PSTVWEDGDY | 540 |
| YRKQLSPLEG GGWLVASGST VAMTEQLQMG FGITVQYGTD TNSVCPKLEF ANDTKIASQL | 600 |
| GNCVEYSLYG VSGRGVFQNC TAVGVRQQRF VYDAYQNLVG YYSDDGNYYC LRACVSVPVS | 660 |
| VIYDKETKTH ATLFGSVACE HISSTMSQYS RSTRSMLKRR DSTYGPLQTP VGCVLGLVNS | 720 |
| SLFVEDCKLP LGQSLCALPD TPSTLTPRSV RSVPGEMRLA SIAFNHPIQV DQLNSSYFKL | 780 |
| SIPTNFSFGV TQEYIQTTIQ KVTVDCKQYV CNGFQKCEQL LREYGQFCSK INQALHGANL | 840 |
| RQDDSVRNLF ASVKSSQSSP IIPGFGGDFN LTLLEPVSIS TGSRSARSAI EDLLFDKVTI | 900 |
| ADPGYMQGYD DCMQQGPASA RDLICAQYVA GYKVLPPLMD VNMEAAYTSS LLGSIAGVGW | 960 |
| TAGLSSFAAI PFAQSIFYRL NGVGITQQVL SENQKLIANK FNQALGAMQT GFTTTNEAFR | 1020 |
| KVQDAVNNNA QALSKLASEL SNTFGAISAS IGDIIQRLDV LEQDAQIDRL INGRLTTLNA | 1080 |
| FVAQQLVRSE SAALSAQLAK DKVNECVKAQ SKRSGFCGQG THIVSFVVNA PNGLYFMHVG | 1140 |
| YYPSNHIEVV SAYGLCDAAN PTNCIAPVNG YFIKTNNTRI VDEWSYTGSS FYAPEPITSL | 1200 |
| NTKYVAPHVT YQNISTNLPP PLLGNSTGID FQDELDEFFK NVSTSIPNFG SLTQINTTLL | 1260 |
| DLTYEMLSLQ QVVKALNESY IDLKELGNYT YYNKWPWYIW LGFIAGLVAL ALCVFFILCC | 1320 |
| TGCGTNCMGK LKCNRCCDRY EEYDLEPHKV HVH | 1353 |

The S1-RBD is located at residues 358 to 558.

In some embodiments, the first reagent may include a nucleocapsid protein of a β-CoV (hereinafter “N protein” or β-CoV N protein”), or antigenic portion thereof that binds an anti-β-CoV antibody. As schematically shown in FIG. 1, the nucleocapsid protein (N-protein) is a structural protein that binds to the coronavirus RNA genome, thus creating a shell (or capsid) around the enclosed nucleic acid. The N-protein also 1) interacts with the viral membrane protein during viral assembly, 2) assists in RNA synthesis and folding, 3) plays a role in virus budding, and 4) affects host cell responses, including cell cycle and translation. The N-protein or antigenic portions thereof may also be used to prepare labeled reagents for use in the present invention.

An exemplary nucleic acid sequence of SARS CoV-2 N protein is provided at NCBI Accession No DQ_243962., version DQ_243962.1, incorporated herein by reference, and reproduced below (SEQ ID NO: 5):

| | |
|---|---|
| 1 | atggctacag tcaaatgggc tgatgcatct gaaccacaac gtggtcgtca gggtagaata |
| 61 | ccttattctc tttatagccc tttgcttgtt gatagtgaac aaccttggaa ggtgatacct |
| 121 | cgtaatttgg tacccatcaa caagaaagac aaaaataagc ttataggcta ttggaatgtt |
| 181 | caaaaacgtt tcagaactag aaagggcaaa cgggtggatt tgtcacccaa gttacatttt |
| 241 | tattatcttg gcacaggacc ccataaagct gcaaaattta gagagcgtgt tgaaggtgtt |
| 301 | gtctgggttg ctgttgatgg tgctaaaact gaacctacag gttacggtgt taggcgcaag |
| 361 | aattcagaac cagagatacc acacttcaat caaaagctcc caaatggtgt tactgttgtt |
| 421 | gaagaacctg actcccgtgc tecttcccgt tctcagtcaa ggtctcagag tcgcggtcgt |
| 481 | ggtgaatcca aatctcaatc tcggaatcct tcaagtgaca gaaaccataa cagtcaggat |
| 541 | gacatcatga aggcagtcgc tgcggctctt aaatctttag gttttgacaa gcctcaggaa |
| 601 | aaagacaaaa agtcagcgaa aacgggtact cctaagcctt ctcgtaatca gagtccttct |
| 661 | tcttttcaat ctgctgccaa gattcttgct cgttctcaga gttctgaaac aaaagaacaa |
| 721 | aagcatgaaa tgcaaaagcc acggtggaaa agacagccta acgatgatgt gacatctaat |
| 781 | gtcacacaat gttttggccc cagagacctt gaccacaact ttggaagtgc aggtgttgtg |
| 841 | gccaatggtg ttaaagctaa aggctatcca caatttgctg agcttgtgcc gtctacagct |
| 901 | gctatgcttt ttgatagtca cattgtttcc aaagagtcag gcaacactgt ggtcttgact |
| 961 | ttcaccacca gagtgactgt gcccaaagac catccacact tgggtaagtt tcttgaggaa |
| 1021 | ttaaatgcat tcactagaga aatgcaacaa cagcctcttc ttaaccctag tgcactagaa |
| 1081 | ttcaacccat cccaaacttc acctgcaact gttgaaccag tgcgtgatga agtttctatt |
| 1141 | gaaactgaca taattgatga agtcaactaa |

Another exemplary SARS CoV-2 N protein has an amino acid sequence provided at NCBI Accession No. AB_B90505, version AB_B90505.1, incorporated herein by reference, and reproduced below (SEQ ID NO: 6):

| | |
|---|---|
| 1 | matvkwadas epqrgrqgri pyslyspllv dseqpwkvip rnlvpinkkd knkligywnv |
| 61 | qkrfrtrkgk rvdlspklhf yylgtgphka akfrervegv vwvavdgakt eptgygvrrk |
| 121 | nsepeiphfn qklpngvtvv eepdsrapsr sqsrsqsrgr gesksqsrnp ssdrnhnsqd |
| 181 | dimkavaaal kslgfdkpqe kdkksaktgt pkpsrnqsps sfqsaakila rsqssetkeq |
| 241 | khemqkprwk rqpnddvtsn vtqcfgprdl dhnfgsagvv angvkakgyp qfaelvpsta |
| 301 | amlfdshivs kesgntvvlt fttrvtvpkd hphlgkflee Inaftremqq qpllnpsale |
| 361 | fnpsqtspat vepvrdevsi etdiidevn |

Another SARS CoV-2 N protein has an amino acid sequence described in Djukic, et al., Virology 557:15-22 (2021), incorporated herein by reference, and reproduced below (SEQ ID NO: 7):

| | |
|---|---|
| MSDNGPQNQR NAPRITFGGP SDSTGSNQNG ERSGARSKQR RPQGLPNNTA SWFTALTQHG | 60 |
| KEDLKFPRGQ GVPINTNSSP DDQIGYYRRA TRRIRGGDGK MKDLSPRWYF YYLGTGPEAG | 120 |
| LPYGANKDGI IWVATEGALN TPKDHIGTRN PANNAAIVLQ LPQGTTLPKG FYAEGSRGGS | 180 |
| QASSRSSSRS RNSSRNSTPG SSRGTSPARM AGNGGDAALA LLLLDRLNQL ESKMSGKGQQ | 240 |
| QQGQTVTKKS AAEASKKPRQ KRTATKAYNV TQAFGRRGPE QTQGNFGDQE LIRQGTDYKH | 300 |
| WPQIAQFAPS ASAFFGMSRI GMEVTPSGTW LTYTGAIKLD DKDPNFKDQV ILLNKHIDAY | 360 |
| KTFPPTEPKK DKKKKADETQ ALPQRQKKQQ TVTLLPAADL DDFSKQLQQS MSSADSTQA | 419 |

In some embodiments, the first reagent includes a fragment of a full-length N protein that binds an anti-β-CoV antibody. A representative fragment includes amino acid residues 58-419 of SED ID NO: 7, reproduced below (SEQ ID NO: 8):

| | |
|---|---|
| QHGKEDLKFP RGQGVPINTN SSPDDQIGYY RRATRRIRGG DGKMKDLSPR WYFYYLGTGP | 60 |
| EAGLPYGANK DGIIWVATEG ALNTPKDHIG TRNPANNAAI VLQLPQGTTL PKGFYAEGSR | 120 |
| GGSQASSRSS SRSRNSSRNS TPGSSRGTSP ARMAGNGGDA ALALLLLDRL NQLESKMSGK | 180 |
| GQQQQGQTVT KKSAAEASKK PRQKRTATKA YNVTQAFGRR GPEQTQGNFG DQELIRQGTD | 240 |
| YKHWPQIAQF APSASAFFGM SRIGMEVTPS GTWLTYTGAI KLDDKDPNFK DQVILLNKHI | 300 |
| DAYKTFPPTE PKKDKKKKAD ETQALPQRQK KQQTVTLLPA ADLDDFSKQL QQSMSSADST | 360 |

As shown in FIG. 1, yet other β-CoV antigens or portions thereof that may be useful in preparing reagents suitable for use in the present invention include the envelope protein (E), the membrane protein (M) and the Hemagglutinin-esterase dimer protein (HE). The E protein is found in small quantities in within the virus. It is believed to be a transmembrane protein and with ion channel activity. The protein facilitates assembly and release of the virus and has other functions such as ion channel activity. It is believed unnecessary for viral replication but necessary for pathogenesis. The M protein is the most abundant structural protein. It does not contain signal sequence and exists as a dimer in the virion. It may have two different conformations to enable it to promote membrane curvature as well as bind to nucleocapsid. The HE protein is present in a subset of betacoronaviruses. The protein binds sialic acids on surface glycoproteins. The protein activities are thought to enhance S protein-mediated cell entry and virus spread through the mucosa.

β-CoV antigens (e.g., in recombinant form) for preparing labeled reagents for use in the present invention are commercially available, e.g., from the Native Antigen Company (SARS-CoV-2 Spike Glycoprotein (S1), Sheep Fc-Tag (HEK293) and SARS-CoV-2 Spike Glycoprotein (S2), Sheep Fc-Tag (HEK293), Sino Biological (ex., SARS-CoV-2 (2019-nCoV) Spike S1(D614G)-His Recombinant Protein, HPLC-verified, SARS-CoV-2 (2019-nCoV) Spike RBD-His (K458R) Recombinant Protein), AcrobioSystem (full-length N protein, Cat. NUN-C81Q6), and Millipore-Sigma (e.g., recombinant SARS CoV-2 N protein fragment (CAT. AXX841)).

### Second Reagents

As disclosed above, the second reagent may be a differentially labeled version of the first reagent.

In other embodiments, the second reagent is a secondary antibody that binds the anti-β-CoV antibody that may be present in a patient sample. The choice of secondary antibody and its origin are not critical provided that the antibody is capable of specifically binding a human, anti-β-CoV antibody that may be present in a patient sample. The antibodies may be of another species such as goat or rodent (e.g., mouse). Representative examples of secondary antibodies include goat anti-human IgG, anti-human IgM and anti-human IgA antibodies, as well as goat anti-human IgA1, anti-human IgA2 antibodies, are available from numerous commercial sources, e.g., from Bethyl Laboratories, Inc. (Anti-IgG: A80-104A; Anti-IgM: A80-100A; Anti-IgA: A80-102A). Labeled antibodies may also be obtained commercially.

In other embodiments, the second reagent includes a nanobody that binds the anti-β-CoV antibody that may be present in a patient sample. Nanobodies are a class of antigen-binding protein derived from camelids that achieve comparable binding affinities and specificities to classical antibodies, despite comprising only a single 15 kDa variable domain. See, Mitchell, et al., Proteins 86(7):697-706 (2018). Nanobodies are also known as single domain antibodies, consist of the heavy chain of the variable region of a camelid antibody, and are of the form VHH (VHH). Since sdAbs can be raised against unique epitope targets inaccessible by conventional antibodies, they offer the ability for precision structural analysis through enhanced molecular and tissue penetration with high affinity and specificity. In some embodiments, the nanobody is an anti-human IgG nanobody (e.g., AF488-anti-IgG-Nanobody, commercially available from Chromotek, as Nano-Secondary® Alpaca anti-human IgG).

### TR-FRET and Donor and Acceptor Fluorophores

TR-FRET is a combination of time-resolved fluorescence (TRF) and FRET. TRF reduces background fluorescence by delaying reading the fluorescent signal, for example, by about 50-200 microseconds. Following this delay (i.e., the gating period), the longer-lasting fluorescence in the sample is measured. Using TR-FRET, interfering background fluorescence due to interfering substances in the sample, for example, is not co-detected. Only the fluorescence generated or suppressed by the energy transfer is measured. The resulting fluorescence of the TR-FRET system is determined by means of appropriate measuring devices. Such time-resolved detection systems use, for example, pulsed laser diodes, light emitting diodes (LEDs) or pulsed dye lasers as the excitation light source. The measurement occurs after an appropriate time delay, i.e., after the interfering background signals have decayed. Devices and methods for determining time-resolved FRET signals are described in the art.

TR-FRET requires that the signal of interest must correspond to a compound with a long fluorescent lifetime. Criteria for selecting an appropriate A TR-FRET donor and acceptor pair include one or more of the following: (1) the emission spectrum of the FRET energy donor should overlap with the excitation spectrum of the FRET energy acceptor; (2) the emission spectra of the FRET partners (i.e., the FRET energy donor and the FRET energy acceptor) should show non-overlapping fluorescence; (3) the FRET quantum yield (i.e., the energy transferred from the FRET donor to the FRET acceptor) should be as high as possible (for example, FRET should have about a 1-100%, e.g., a 1%, 5%, 10%, 20%, 30%, 40%, 50%, 60%, 70%, 80%, 85%, 90%, 95%, 98%, and 99% efficiency over a measured distance, of 1-20 nm, e.g., 5-10 nm); (4) the FRET signal (i.e., fluorescence) must be distinguishable from fluorescence produced by the sample, e.g., autofluorescence; and (5) the FRET donor and the FRET acceptor should have half-lives that allow detection of the FRET signal (e.g., TR-FRET can be bright and can occur on a timescale ranging from 10-9 seconds to 10-3 seconds).

Donor/acceptor fluorophore pairs for use in TR-FRET-based assays are known in the art. See, e.g., Joseph R. Lakowicz (Principles of fluorescence spectroscopy, 2nd edition, Kluwer academic/plenum publishers, NY (1999)).

Donor fluorophores advantageously emit long-lived fluorescence, typically in the order of >0.1 milliseconds (ms), preferably between 0.5 and 6 ms). In this fashion, excitation of the donor fluorophore by a pulsed light source (such as a flash lamp), followed by a delay and then FRET signal measurement (known in the art as a counting window) allows short-lived fluorescence to subside before the measurement is made. This property enables the assay to be conducted in a time-resolved manner which reduces background (signal-to-noise ratios) and in turn, enhances sensitivity and accuracy.

Representative examples of types of donor fluorophores include lanthanide metals and complexes thereof, including chelates and cryptates. Exemplary lanthanides include terbium (Tb), europium (Eu), dysprosium (Dy), samarium (Sm), neodymium (Nd), ytterbium (Yb), erbium (Er), and their respective 3+ complexes. Such complexes include cryptates and chelates, representative examples of which are described, for example in U.S. Patent Application Publication 2015/0198602 A1, incorporated herein by reference. In some embodiments, the donor fluorophore is terbium or europium, or a cryptate or chelate thereof, examples of which are described the '602 Patent Publication. These donor fluorophores are commercially available, e.g., from Cisbio. Eu3+, for example, has a fluorescent lifetime in the order of milliseconds.

Representative examples of acceptor fluorophores include allophycocyanins (tradename XL665); luminescent organic molecules, such as rhodamines, cyanines (e.g., Cy5), squaraines, coumarins, proflavins, acridines, fluoresceins, boron-dipyrromethene derivatives (commercially available under the tradename “BODIPY”), fluorophores known under the name “Atto”, fluorophores known under the name “DY”, compounds known under the name “Alexa”, and nitrobenzoxadiazole.

The “Alexa” compounds are commercially available, e.g., from Invitrogen; the “Atto” compounds are commercially available from Atto-tec; the “DY” compounds are commercially available from Dyomics; and the “Cy” compounds are commercially available from Amersham Biosciences.

Table 1 lists representative examples of donor/acceptor pairs for TR-FRET/HTRF¹, while Table 2 lists excitation and emission (nm) of known FRET fluorophores.

TABLE 1

| Donor | Acceptor | R₀ (Å)² |
|---|---|---|
| Fluorescein | Tetramethylrhodamine | 55 |
| IAEDANS | Fluorescein | 46 |
| EDANS | Dabcyl | 33 |
| BODIPY FL | BODIPY FL | 57 |
| Fluorescein | QSY 7 and QSY 9 dyes | 61 |
| Alexa Fluor 488 | Alexa Fluor 555 | 70 |
| Alexa Fluor 594 | Alexa Fluor 647 | 85 |
| Europium (III) cryptate | XL665 (allophycocyanine) or d2 | 90 |
| Terbium (III) | Fluorescein | 70 |

¹Adapted from Invitrogen.com (FRET; Alexa dyes) and Cysbio (TR-FRET);
²Ro is the distance at which FRET efficiency is 50%. The excitation and emission of various donor and acceptor fluorophores that may be useful in practicing the present invention are described in U.S. Patent Application Publication 20180356411 A1.

TABLE 2

| FLUOROPHORE | Excitation (nm) | Emission (nm) |
|---|---|---|
| 5-Carboxynapthofluorescein | 512/598 | 563/668 |
| 5-ROX (carboxy-X- | 578 | 604 |
| rhodamine) | | |
| | 567 | 591 |
| Alexa Fluor 568 TM | 577 | 603 |
| Alexa Fluor 594 TM | 590 | 617 |
| | 594 | 618 |
| Alexa Fluor 633 TM | 632 | 650 |
| Alexa Fluor 647′m | 647 | 666 |
| Alexa Fluor 660 TM | 668 | 698 |
| Alexa Fluor 680 TM | 679 | 702 |
| Allophycocyanin (APC) | 630-645 | 655-665 |
| APC-Cy7 | 625-650 | 755 |
| BOBO TM -3 | 570 | 602 |
| Bodipy | 492-591 | 509-676 |
| Bodipy TR | 589 | 617 |
| Bodipy TR ATP | 591 | 620 |
| Calcium Crimson TM | 588 | 611 |
| | 589 | 615 |
| Carboxy-X-rhodam ne (5- | 576 | 601 |
| ROX) | | |
| Cy3.5 TM | 581 | 598 |
| Cy5.1 8 | 649 | 666 |
| Cy5.5 TM | 675 | 695 |
| Cy5 TM | 649 | 666 |
| Cy7 TM 710, 743 767, 805 | 710 | 767 |
| | 743 | 805 |
| Dysprosium | 305-335 | 465-495 |
| | | 565-595 |
| Europium | 315-350 | 600-635 |
| | | 675-715 |
| Europium (III) chloride | 315-350 | 600-635 |
| | | 675-715 |
| FL-645 | 615-625 | 665 |
| Fura Red TM (high pH) | 572 | 657 |
| LaserPro | 795 | 812 |
| Samarium | 325-355 | 475-505 |
| | | 545-575 |
| | | 585-615 |
| | | 630-660 |
| SureLight CO | 640-660 | 660-680 |
| Terbium | 305-335 | 475-505 |
| | | 530-560 |
| | | 570-600 |
| | | 605-635 |
| Texas Red TM | 595 | 620 |
| Texas Red-X TM conjugate | 595 | 615 |
| Thiadicarbocyanine (DiSC3) | 651 | 674 |
| | 653 | 675 |
| Thiazine Red R | 596 | 615 |
| TO-PRO-1 | 515 | 531 |
| TO-PRO-3 | 644 | 657 |
| TO-PRO-5 | 747 | 770 |
| TOTO-3 | 642 | 660 |
| ULight | 630-655 | 655-675 |
| Ultralife | 656 | 678 |
| X-Rhodamine | 580 | 605 |
| XRITC | 582 | 601 |
| YO-PRO-3 | 613 | 629 |

See, U.S. Patent Application Publication 20180356411 A1.

In some embodiments of the present invention, the donor fluorophore is terbium (Tb) or europium, or a cryptate or chelate thereof, and the fluorophore acceptor is an organoboron fluorescent dye, e.g., boron-dipyrromethene (4,4-difluoro-4-bora-3 a,4a-diaza-s-indacene)(commercially available under the tradename BODIPY™), sodium 6-amino-9-(5-((aminomethyl)carbamoyl)-2-carboxyphenyl)-3 -iminio-3H-xanthene-4,5-disulfonate (commercially available under the tradename Alexa488™, and 2-[5-[3,3-dimethyl-5-sulfo-1-(3-sulfopropyl)indol-1-ium-2-yl]penta-2,4-dienylidene]-3-methyl-3-[5-oxo-5-(6-phosphonooxyhexylamino)pentyl]-1-(3-sulfopropyl)indole-5-sulfonic acid (commercially available under the tradename Alexa647™).

In some embodiments, the fluorophore donor/acceptor pair is Tb and BODIPY. In some embodiments, the fluorophore donor/acceptor pair is Eu and ALEXA647, respectively.

### Labeling of the Reagents with Donor and Acceptor Fluorophores

The art teaches how to proteinaceous entities with donor and acceptor fluorophores in accordance with a variety of techniques and coupling agents. See, e.g., the '602 Patent Publication. Commercially available kits are also available for this purpose. For example, a kit, commercially available from Cisbio and Perkin Elmer, allows for labeling peptides, proteins and oligonucleotides with Terbium cryptate, which includes N-hydroxysuccinimide-activated Terbium-Trisbipyridine (TBP).

The reagents are differentially labeled with the fluorophore donor and acceptor. The respective molar concentrations for any given pair of fluorophore donor and acceptor in an inventive TR-FRET assay are determined to enhance the FRET signal and facilitate its detection. As such, the molar concentrations may vary, depending upon any given pair of fluorophore donor and acceptors, and the proteinaceous portions of the reagents that will carry them. Determining the relative molar concentrations of the labels for use with any given β-CoV antigen (e.g., SARS CoV-2 antigen) and portion of the second reagent (e.g., secondary antibody or nanobody) so as to optimize the FRET signal and minimize background noise, is within the level of skill in the art. The working examples illustrate optimization of these molar concentrations using techniques known in the art.

In some embodiments, for example, a concentration of a donor fluorophore such as Terbium or Europium (e.g., as a label for a SARS CoV-2 antigen) within the range of about 1.75 nM to about 30 nM (relative to a TR-FRET assay volume of 15 μL) may be useful. Concentrations outside this range, both lower and higher, may also be useful. In some embodiments, the concentration of a donor fluorophore such as Tb or Eu is about 7.5 nM, and in other embodiments, the concentration is about 15 nM.

In some embodiments, a concentration of an acceptor fluorophore, such as BODIPY (e.g., when used with Tb as the fluorophore donor) of about 50 nM-1 μM (relative to a TR-FRET assay volume of 15 μL) may be useful. Concentrations outside this range, both lower and higher, may also be useful. In some embodiments, the concentration of BODIY is about 250 nM (relative to a TR-FRET assay volume of 15 μL).

The optimal molar concentrations of the fluorophore donor and acceptor relative to one another may depend on Degree of Labeling (DoL). As is known in the art, the DoL is the average number of labels (which in this case are the fluorophore donor and acceptor) coupled to a protein molecule (which in this case are the SARS-CoV-2 antigenic proteins or antigenic fragments thereof and the antibodies). As in the case of the molar concentrations, the DoL may vary. Determining the relative DoLs of the labels for use with any given SARS CoV-2 antigen and secondary antibody so as to optimize the FRET signal and minimize background noise, is within the level of skill in the art. In the present methods, the DoL, e.g., with respect to Tb) is generally in the range of about 1.0 to about 3.8. In some embodiments, the DoL is within the range of about 1.8 to about 3.8. In some embodiments, the DoL is about 1.8. DoL values outside this range, both lower and higher, may also be useful. However, a DoL of about 8 (and higher) for Tb might be disadvantageous in that the FRET signal is too strong to be practical. The working examples illustrate optimization of a DoL for Tb using techniques known in the art. The DoL for a Tb-anti-IgG-Nanobody is about 1. As demonstrated in the working examples, DoL may be determined in accordance with standard techniques.

### Patients and Patient Samples

The present methods entail testing body fluid samples obtained from individuals. The samples may include whole blood or a component thereof such as serum and plasma, saliva, and tears. In some embodiments, the body fluid sample is serum or plasma. In some embodiments, the body fluid sample is dried whole blood. Practice of the invention is not limited to any subpopulations of individuals. Samples may be obtained from any individual (patient), and not just individuals who exhibited symptoms of the infection. Individuals who desire, believe to be in need of, who have been required to be tested for β-CoV (e.g., SARS-CoV-2), and/or are asymptomatic may be tested.

### Practice of the Assay Methods

Common to the inventive methods entails detecting anti-β-CoV antibodies (e.g., SARS CoV-2 antibodies) by inducing a proximity event that can be detected through TR-FRET signals. Choice and optimization of standard reagents and solutions, concentrations of reagents and apparati (e.g., a fluorometer, microplates, excitation means, donor and acceptor fluorophores, and measurement of signals and other parameters involved in TR-FRET-based homogeneous assay formats for antibody detection are within the level of skill in the art. See, e.g., Saraheimo S, Hepojoki J, Nurmi V, Lahtinen A, Hemmilä I, Vaheri A, et al. (2013) Time-Resolved FRET-Based Approach for Antibody Detection—A New Serodiagnostic Concept. PLoS ONE 8(5): e62739; doi.org/10.1371/journal.pone.0062739 (and references cited therein).

Broadly, the present invention provides a homogeneous, TR-FRET-based method for detection of β-CoV antibodies (e.g., SARS-CoV-2 antibodies) in a patient fluid sample. The β-CoV antibodies that are detected by the present methods are referred to herein as primary antibodies. To test a body fluid sample from a patient, the inventive methods employ two reagents, each of which binds to an anti-SARS CoV-2 antibody. At least one of the reagents is a TR-FRET labeled β-CoV antigen (e.g., a SARS CoV-2 antigen), which as used herein, refers to any β-CoV protein or portion thereof that is capable of eliciting an antibody response in a patient infected with a β-CoV such as SARS-CoV-2. In some embodiments, the second reagent is the same β-CoV antigen but with the complementary TR-FRET label. In some embodiments, the second reagent is a TR-FRET labeled secondary antibody that binds to the primary antibody. In some embodiments, the second reagent is a TR-FRET labeled nanobody that binds to the primary antibody. The reagents are differentially labeled with a donor fluorophore and an acceptor fluorophore. The body fluid sample is brought into contact with the two, differentially labeled reagents in a homogeneous assay format. Due to the multi-valent properties of antibodies in general, an anti-β-CoV antibody (e.g., anti-SARS-CoV-2 antibody) present in the fluid sample will bind the differentially labeled reagents. If anti-β-CoV antibodies (e.g., SARS-CoV-2 antibodies) are present in the sample, they will be bound by the first and second reagents, bringing the donor and acceptor fluorophores into close proximity. These binding events generate a detectable FRET signal, diagnostic of anti-β-CoV antibodies (e.g., anti-SARS-CoV-2 antibodies) and infection with a β-CoV virus. Conversely, the lack of a FRET signal indicates an absence of anti-β-CoV antibodies (e.g., anti-SARS CoV-2 antibodies) and no infection with the β-CoV virus.

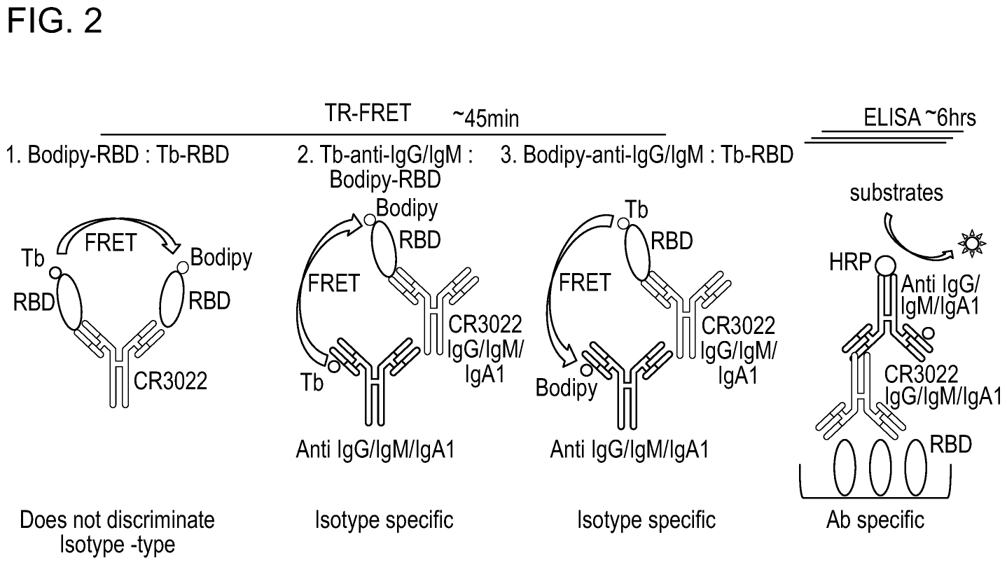

In some embodiments, the methods are designed to detect presence of anti-β-CoV antibodies (e.g., anti-SARS-CoV-2 antibodies), without regard to their class or subtype. A representative TR-FRET-based homogeneous assay format is schematically illustrated in FIG. 2 (assay 1). A patient fluid sample such as plasma or serum is contacted with a SARS-CoV-2 antigen differentially labeled with a donor fluorophore and an acceptor fluorophore. Therefore, in these embodiments, the SARS-CoV-2 antigen labeled with the donor fluorophore is considered as the first reagent. The SARS-CoV-2 antigen labeled with the acceptor fluorophore is considered as the second reagent. The body fluid sample is brought into contact with the differentially labeled reagents in a homogeneous assay format. Due to the multivalent nature of antibodies in general, if SARS-CoV-2 antibodies are present in the sample, they will bind with the first and second reagents, bringing the donor and acceptor fluorophores into close proximity in the Fab region of the SARS-CoV-2 antibody, resulting in generation of a FRET signal. As shown in FIG. 2 (assay 1), one Fab arm of the antibody will bind with the SARS-CoV-2 antigen labeled with the donor fluorophore and the other Fab arm of the antibody will bind with the SARS-CoV-2 antigen labeled with the acceptor fluorophore. Detection of the FRET signal indicates presence of anti-SARS-CoV-2 antibodies in the fluid sample, and hence diagnosis of SARS-CoV-2 infection.

These embodiments detect the presence of anti-β-CoV antibodies (e.g., anti-SARS-CoV-2 antibodies). They are not specific as to which class (e.g., IgG, IgM and/or IgA) or subtype (e.g., IgA1, IgA2 etc.) the anti-β-CoV antibodies (e.g., anti-SARS-CoV-2 antibodies) belong. The present methods offer additional levels of specificity to detect these kinds of anti-β-CoV antibodies (e.g., anti-SARS-CoV-2 antibodies).

Accordingly, in other embodiments, the methods specific classes or subtypes of anti-β-CoV antibodies (e.g., anti-SARS-CoV-2 antibodies) present in a patient fluid sample. Two representative TR-FRET-based homogeneous assay formats are schematically illustrated in FIG. 2, assays 2 and 3. To achieve this additional level of specificity, the reagents are selected such that the first reagent is a SARS-CoV-2 antigen, and the second reagent is the secondary antibody that is capable of binding to the primary, anti-SARS-CoV-2 antibody. The two reagents are differentially labeled with the donor and acceptor fluorophores. Therefore, when the SARS-CoV-2 antigen is labeled with the donor fluorophore, the secondary antibody is labeled with the acceptor fluorophore (assay 3), and vice-versa (assay 2). For purposes of detecting different classes of anti-SARS-CoV-2 antibodies, the secondary antibody does not have to be a human antibody. It may originate from any non-human species such as a goat or rodent (e.g., mouse) so long as it specifically detects any class of the human anti-SARS CoV-2 antibodies generated by the patient being tested. In some embodiments, the secondary antibody may be a standard human, anti-IgG, anti-IgM or anti-IgA antibody. The same principles apply to detecting different subtypes of anti-SARS-CoV-2 antibodies. The secondary antibody does not have to be a human antibody so long as it is capable of binding to and detecting any subtypes of the human anti-SARS CoV-2 antibodies generated by the patient being tested. Accordingly, the secondary antibody may be a standard human, anti-IgA1, or anti-IgA2 antibody.

The body fluid sample is brought into contact with the differentially labeled reagents in the homogeneous assay format. If SARS-CoV-2 antibodies of a specific class or subtype are present in the sample, they will be bound by the first and second reagents, bringing the donor and acceptor fluorophores into close proximity, resulting in generation of a FRET signal. As shown in FIG. 2 (assay 2), a Fab arm of the antibody will bind with the SARS-CoV-2 antigen labeled with the acceptor fluorophore and due to the affinity of the secondary antibody labeled with the donor fluorophore, it will bind the Fc region of the primary antibody. As shown in FIG. 2 (assay 3), a Fab arm of the antibody will bind with the SARS-CoV-2 antigen labeled with the donor fluorophore and the Fc region of the antibody will bind with the secondary antibody labeled with the acceptor fluorophore. Detection of the FRET signal indicates presence of anti-SARS-CoV-2 antibodies of a specific class or subtype in the fluid sample, and hence diagnosis of SARS-CoV-2 infection.

These various embodiments have been illustrated using terbium and BODIPY as the donor and acceptor fluorophores, respectively. As disclosed herein, the methods can be practiced with other FRET donor/acceptor fluorophore pairs that are capable of generating a detectable FRET signal based on the distances at hand. Such FRET donor/acceptor pairs include Eu and ALEXA647. Table 1 lists representative examples of donor/acceptor pairs for TR-FRET/HTRF, while Table 2 lists excitation and emission (nm) of known FRET fluorophores.

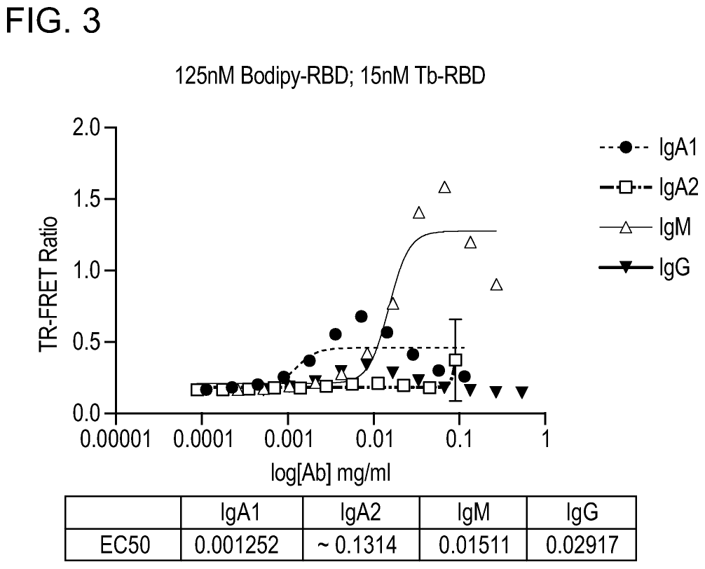

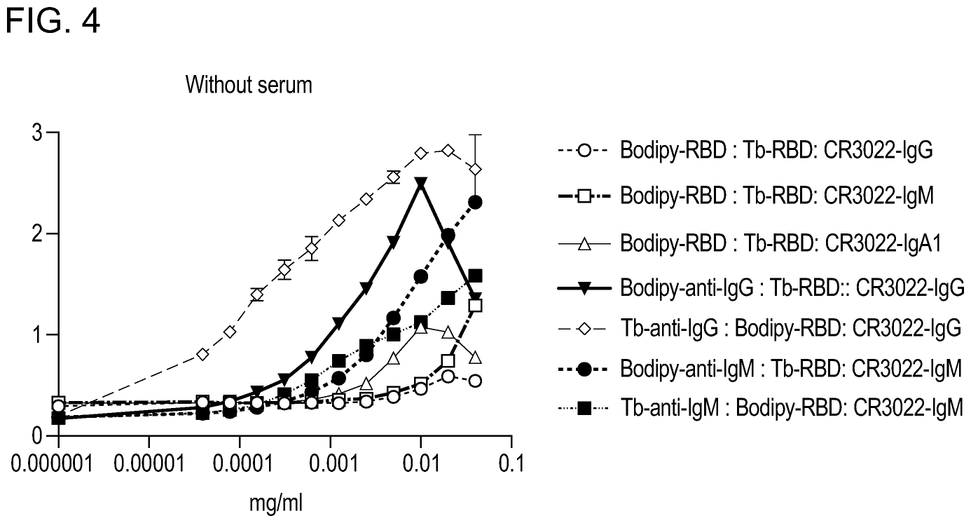

To obtain isotype specific detection, either the donor or the acceptor fluorophore can be moved to a secondary antibody (assay 2 and assay 3 shown in FIG. 2) or a nanobody which will bind to the antigen specific IgG/IgM/IgA present in the patient sample. When antigen binds to the primary antibody, and the secondary antibody or nanobody recognizes the primary antibody, the donor and acceptor fluorophores are brought into close proximity, resulting in a positive TR-FRET signal (FIG. 3 and FIG. 4).

The assay methods may be conducted with multiple (2 or more) pairs of labeled reagents, provided that the FRET signals generated from any one FRET donor/acceptor pair are substantially non-overlapping with all other FRET donor/acceptor pairs.

### Types of FRET Signals and Measurement/Detection Thereof

“FRET signal” refers to any measurable signal representative of FRET between the fluorescent donor compound and the acceptor compound. A FRET signal may therefore be a change in the intensity or lifetime of luminescence of the fluorescent donor compound or of the acceptor compound. Any of a variety of light-emitting and light-detecting instruments can be used to initiate FRET (e.g., excite the donor fluorophore or excite a reagent capable of exciting the donor fluorophore) and/or detect the emission produced. The light emissions produced by donor and acceptor fluorophores, i.e., the FRET signal, can be detected or measured visually, photographically, actinometrically, spectrophotometrically, or by any other convenient means, such as with the use of a fluorometer. See, e.g., Saraheimo, supra.

The binding of the antibody or ligand to the target antigen can be determined qualitatively, i.e., by the presence or absence of the FRET signal; with the absence of any FRET signal being indicative of no binding. Usually the “absence of a FRET signal” is defined by a certain threshold, i.e., after deduction of any background signal. The background signal is usually determined by performing the FRET assay with all reagents but the antibody or ligand to be tested.

The binding of the labeled reagents to the target anti-β-CoV antibodies (e.g., anti-SARS-CoV-2 antibodies) can be determined qualitatively or quantitatively. Qualitative determinations simply detect presence or absence of the FRET signal. Absence of any FRET signal indicates no binding. Usually the “absence of a FRET signal” is defined by a certain threshold, i.e., after deduction of any background signal. The background signal is usually determined by performing the FRET assay with all assay reagents except for the labeled reagents. For quantitative determinations, level or strength of binding can be determined by testing the labeled reagents in different concentrations which yields half maximal effective concentration (EC₅₀). EC₅₀ refers to the concentration of the labeled reagent at which binding (with the anti-β-CoV antibodies (e.g., anti-SARS-CoV-2 antibodies) is halfway between the baseline and maximum after a specified exposure time. The EC₅₀ dose response curve can be generated.

The disclosed reagents may be conveniently packaged in an assay kit, to facilitate practice of the homogeneous, TR-FRET-based method for detection of anti-β-CoV antibodies (e.g., anti-SARS-CoV-2 antibodies) in a patient fluid sample.

A further aspect of the present invention is directed to an assay kit for homogeneous, TR-FRET-based method for detection of anti-β-CoV antibodies (e.g., SARS-CoV-2 antibodies) in a patient fluid sample. Broadly, the kits may include the first and second reagents, disposed in the same or different containers, and printed instructions for carrying out the assay method.

In some embodiments, the first and second reagents include a first subpopulation of a β-CoV antigen (e.g., a SARS-CoV-2 antigen) and a second subpopulation of the β-CoV antigen, respectively, wherein the first and second subpopulations are differentially labeled with a donor fluorophore and an acceptor fluorophore, wherein the first and second reagents may be disposed in the same or different containers. The first reagent may include the donor fluorophore in which case the second reagent may include the acceptor fluorophore, and vice versa.

In other embodiments, the first reagent may include a β-CoV antigen (e.g., a SARS-CoV-2 antigen) and the second reagent may include at least one secondary antibody or a nanobody that binds the anti-β-CoV antibody. The at least one secondary antibody or the nanobody may bind a specific class or subtype of human antibodies. The first and second reagents are differentially labeled with the donor fluorophore and the acceptor fluorophore. The first and second reagents differentially labeled with the fluorophore donor and acceptor fluorophore may be disposed in separate containers.

The printed instructions describe use of the reagents, along with any necessary instrumentation, in the homogeneous, TR-FRET-based method for detection of anti-β-CoV antibodies (e.g., SARS-CoV-2 antibodies) in a patient fluid sample.

Practice of embodiments illustrated in FIG. 2 (assay 1) entails use of the first and second reagents that include first and second subpopulations of a β-CoV antigen (e.g., a SARS-CoV-2 antigen) that are differentially labeled with a donor fluorophore and an acceptor fluorophore. For this purpose, they may be disposed in the same container.

Practice of embodiments illustrated in FIG. 2 (assays 2 and 3) entails use of differentially labeled first and second reagents wherein the first reagent includes a β-CoV antigen (e.g., a SARS-CoV-2 antigen) and the second reagent includes at least one secondary antibody or a nanobody that binds the anti-β-CoV antibody. Hence, providing them in separate containers provides greater flexibility as to the types of assay formats that can be performed with the components of the kit. For example, subpopulations of secondary antibodies or nanobodies differentially labeled with the donor fluorophore and the acceptor fluorophore are not used in the same assay. Hence, providing a first subpopulation of the at least one secondary antibody or nanobody labeled with the donor fluorophore disposed in a first container, and a second subpopulation of the at least one secondary antibody or nanobody labeled with the acceptor fluorophore disposed in a second container provides the minimum necessary reagents to practice the disclosed methods throughout their entire scope. In this regard, the kit may further include second, third, etc. labeled secondary antibodies.

Therapy might be in order, depending on the results of the test. Therefore, in some embodiments, the patient tested might be treated with a prophylactic (vaccine) or an anti-β-CoV therapeutic agent (e.g., an anti-SARS-CoV-2 therapeutic agent). For example, if the results are negative (no anti-β-CoV antibodies), a vaccine (prophylactic) might be prescribed. If the results are positive and the patient is symptomatic, therapy might be in order. If the results are positive and the patient is asymptomatic (which could mean recent infection, but active phase has subsided, or patient with active phase but who simply doesn't manifest typical symptoms), vaccine or therapy might be in order.

### EXAMPLES

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1: Materials and Methods

### Antigen production

The full-length Spike protein of SARS-CoV-2 (S protein prefusion stabilized with furin site removed, expressed in TunaCHO) was purchased from Lake Pharma (Cat. 46328) and full-length N protein of SARS-CoV-2 (construct 1-419) was purchased from AcrobioSystem (Cat. NUN-C81Q6). RBD protein was purchased from LakePharma (Cat. 46438). Full-length Spike protein of SARS-CoV (Cat. 40634-V08B) and MERS-CoV (Cat. 40069-V08B) were purchased from Sino Biological.

### Constructs and Protein Purification of CR3022 Antibodies

CR3022 IgG, IgM, IgA1 antibodies were expressed in Expi293T cells according to the manufacturer's protocol (Thermo Fischer Scientific, A14525) using transfection ratios of 1:1 or 2:1 of heavy to light chain. The cell suspension was cleared using centrifugation, 15 minutes at 46500 relative centrifugal force (rcf) (Ti45, Beckman Coulter). The clarified media was filtered with a 0.45 μm filter before adding to binding columns pre-equilibrated with binding buffer (PBS, 10 mM Na₂HPO₄, 1.8 mM KH₂PO₄, 137 mM NaCl, 2.7 mM KCl at pH 7.4) and using protein G (GE, GE17-0405-01) for IgG, protein L (GE, GE17-5478-15) for IgM or peptide M (InvivoGen, gel-pdm-5). The beads were washed with 20-50 column volumes (CV) of binding buffer. The protein was eluted from the beads with 6-15 CV of 0.1 M glycine pH 3.0 elution buffer and immediately quenched using a 10:1 ratio of 1 M Tris-HClpH 8.0. The protein-containing fractions were pooled and flash-frozen in liquid nitrogen at 0.1-1.5 mg/mL. The antibodies were stored at −80° C. until further use. Concentrations were estimated using Bradford assay.

### Constructs and Protein Purification for Biotinylated SARS-CoV-2 N Protein

The full-length N protein was cloned and expressed in insect cells with N-term Strep-Avi-Tev fusion tag. Cells were lysed by sonication (in 50 mM Tris pH 8.0, 200 mM NaCl, 0.1% Triton X-100, 1 mM PMSF and 1 tablet of complete protease inhibitor cocktail Roche Applied Science), lysate cleared by high-speed centrifugation, and the supernatant passed over StrepTactin-XT HC affinity resin (IBA). Target protein was eluted using biotin and subjected to Poros50HQ ion exchange chromatography. Purification was completed using size exclusion chromatography with a 26/60 Superdex S200 column (GE Healthcare) in 50 mM HEPES pH 7.4, 200 mM NaCl and 2 mM TCEP. The purified avi tagged N protein was biotinylated in presence of BirA enzyme, 10 mM MgCl₂, 2 mM biotin, 20 mM ATP. Biotinylation was confirmed by mass spectrometry. The protein-containing fractions were pooled and flash-frozen in liquid nitrogen at 1.6 mg/mL for N protein. The proteins were stored at −80° C. until further use. Concentrations were estimated using Bradford assay.

### Serum Samples

Serum/plasma samples used in this study were obtained through the Ragon Institute Clinical Services (96w_testset), the Brigham and Women Hospital (BWH set) and Dana-Farber Lung Cancer Center (pre-pandemic negative controls in 96w_testset). All samples were collected after subjects provided signed informed consent or were collected as discarded samples under approved Massachusetts General Hospital (MGH) institutional protocols. The BWH set included sample convalescents with a confirmed prior SARS-CoV-2 RNA+ and two repeat RNA-negative tests after 2 weeks of isolation (CoV2+) and a group of low-risk community members (healthy). Subjects were included if they had a positive SARS-CoV-2 RNA test. Samples were heat inactivated at 60° C. for 1 hour.

### Protein Labeling with NCP311-Tb or Bodipy

Anti-IgG antibody (2.5 mL; Bethyl, A80-104A), anti-IgM antibody (2.5 mL; Bethyl, A80-100A), RBD protein (2.5 mL; LakePharma, 46438) or SARS-CoV (Cat. 40634-V08B) and MERS-CoV (Cat. 40069-V08B) at concentration of 0.25 mg/mL or anti-IgG Nanobody (Chromotek, srbAF488-1-100 was purchased as AF488 labelled sample, and srbGCys2-1-500 for labelling with NCP311-Tb) or S protein (2.5 mL; Lake Pharma, 46328) at a concentration of 1 mg/mL in buffer was exchanged into 100 mM sodium carbonate buffer at pH 8.5, 0.05% TWEEN-20 detergent using PD-10 Desalting Columns (Sigma, GE17-0851-01) according to the manufacturer's protocol with a 0.5 mL per fraction elution. Protein containing fractions were pooled at 0.5-1 mg/mL and the appropriate volume of either NCP311-Tb (1 mM in dimethylacetamide (DMAc)) or BODIPY-NHS (10 mM in DMSO) was added to achieve a molar ratio of approximately 4-5×NCP311-Tb (see, PCT Patent Publication No. WO 2020/086629, incorporated herein by reference) or 3× for the anti-IgG Nanobody with the final degree of labelling for the Nanobody of 1 or 6× BODIPY to antibody. BODIPY-NHS is commercially available from numerous commercial sources, e.g., Thermo Fisher (D2184) and Abcam (ab146451). Alternatives for NCP311-Tb for use in the present invention are commercially available, e.g., from Thermo Fisher (Lanthascreen terbium NETS; PV3578), from CisBio (Terbium cryptate; 62TBSPEA), and Perkin Elmer (DELFIA TRF reagents; AD0009).

### Structure of NCP311-Tb

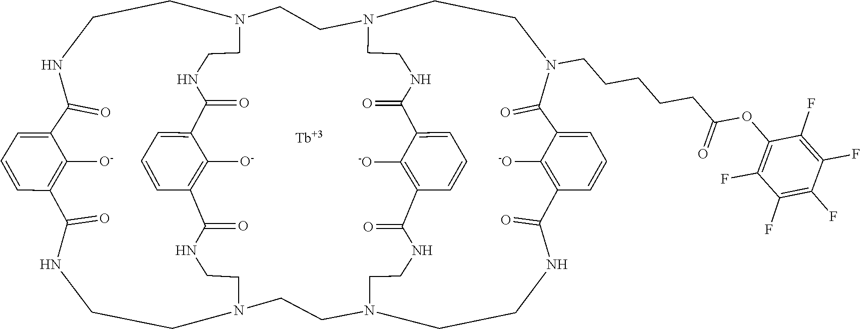

The reaction mixture was briefly vortexed and allowed to stand at room temperature for 1 hour. To purify the labeled conjugates, the labeling reaction was buffer exchanged into 50 mM sodium phosphate buffer pH 7.4, 137 mM NaCl, 0.05% TWEEN-20 detergent using PD-10 desalting columns and following the manufacturer's protocol using 0.5 mL elution fractions. Protein containing fractions were pooled and flash-frozen in liquid nitrogen at 0.4-0.6 mg/mL concentration and stored at −80° C.

The corrected A₂₈₀ value (A_{280,corr}) of protein conjugate was determined via Nanodrop (0.1 cm path length) by measuring A₂₈₀ and A₃₄₀, using equation 1:

A_{280,corr}=A₂₈₀−(A₃₄₀×cf) (1)

where cf is the correction factor for the Tb complex contribution to A₂₈₀ and is equal to 0.157. The concentration of protein conjugate, c_{ab} (M) was determined using equation 2:

$${c}_{ab} = \frac{{A}_{280 , corr}}{ε b} \qquad ( 2 )$$

where ε is the antibody extinction coefficient at A₂₈₀, equal to 210,000 M⁻¹cm⁻¹ for standard IgG classes, 24,075 M⁻¹cm⁻¹ for anti-IgG Nanobody, 80,200 M⁻¹cm⁻¹ for RBD, and 240,000 M⁻¹cm⁻¹ for S protein and b is path length in cm (0.1 cm). The concentration of Tb complex, c_{Tb} (M) covalently bound to the proteins was determined using equation 3:

$${c}_{Tb} = \frac{{A}_{340}}{ε b} \qquad ( 3 )$$

where ε is the complex extinction coefficient at A₃₄₀, equal to 22,000 M⁻¹cm⁻¹ and b is path length in cm (0.1 cm). The degree of labeling (DOL) was calculated using equation 4:

$$DOL = \frac{{c}_{Tb}}{{c}_{ab}} \qquad ( 4 )$$

### TR-FRET Assay for the Receptor Binding Domain

The titration of CR3022 IgG/IgM/IgA1 antibody or the dilution of tested human serum samples was added to assay mix with final concentrations of 15 nM Tb-labeled RBD and 250 nM BODIPY-labeled anti-IgG/IgM/IgA in a buffer containing PBS, 0.05% Tween-20 (Sigma Aldrich P9416). Serum samples were diluted in the buffer containing 50 mM Tris pH 8.0, 140mM NaCl, 0.05% Tween-20 and 1% BSA (Cell Signaling Technology 9998S). TR-FRET assays were performed in a 384-well microplate (Corning, 4514) with 15 μL final assay volume. Before TR-FRET measurements were conducted, the reactions were incubated for 1 hour at room temperature (RT). After excitation of terbium fluorescence at 337 nm, emission at 490 nm (Terbium) and 520 nm (BODIPY) was recorded with a 70 μs delay over 130 μs to reduce background fluorescence and the reaction was followed over >20 or >100 second cycles of each data point using a PHERAstar FS microplate reader (BMG Labtech). The TR-FRET signal of each data point was extracted by calculating the 520/490 nm ratio.

### TR-FRET Assay for Spike Protein of SARS-CoV-2, SARS-CoV or MERS-CoV

The titration of CR3022 IgG/IgM/IgA1 antibody or the dilution of tested human serum samples was added to assay mix with final concentrations of 7.5 nM Tb-labeled S protein of SARS-CoV-2, SARS-CoV or MERS-CoV, and 250 nM BODIPY-labeled anti-IgG/anti-IgM/anti-IgA or AF488-anti-IgG-Nanobody in a buffer containing PBS, 0.05% Tween-20 (Sigma Aldrich P9416). In an alternative setup titrations were performed in the final concentrations of 7.5 nM Tb-anti-IgG-Nanobody, 250 nM BODIPY-S. Serum samples were diluted in the buffer containing 50 mM Tris pH 8.0, 140mM NaCl, 0.05% Tween-20 and 1% BSA (Cell Signaling Technology 9998S). TR-FRET assays were performed in 384-well microplate (Corning, 4514) with 15 μL final assay volume. Before TR-FRET measurements were conducted, the reactions were incubated for 1 hour at RT. After excitation of terbium fluorescence at 337 nm, emission at 490 nm (Terbium) and 520 nm (BODIPY/AF488) was recorded with a 70 μs delay over 130 μs to reduce background fluorescence and the reaction was followed over >20 or >100 second cycles of each data point using a PHERAstar FS microplate reader (BMG Labtech). The TR-FRET signal of each data point was extracted by calculating the 520/490 nm ratio.

### TR-FRET Assay for Nucleocapsid Protein

The dilution of tested human serum samples was added to the assay mix with final concentrations of 20 nM biotinylated N protein, 24 nM Streptavidin-Tb, and 250 nM BODIPY-labeled anti-IgG in a buffer containing PBS, 0.05% Tween-20 (Sigma Aldrich P9416). Serum samples were diluted in buffer containing 50 mM Tris pH 8.0, 140 mM NaCl, 0.05% Tween-20 and 1% BSA (Cell Signaling Technology 9998S). TR-FRET assays were performed in a 384-well microplate (Corning, 4514) with 15 μL final assay volume. Biotinylated N protein and Streptavidin-Tb were premixed and incubated for 10 minutes at RT. Before TR-FRET measurements were conducted, the reactions were incubated for 1 hour at RT. After excitation of terbium fluorescence at 337 nm, emission at 490 nm (Terbium) and 520 nm (BODIPY) was recorded with a 70 μs delay over 130 μs to reduce background fluorescence and the reaction was followed over >20 or >100 second cycles of each data point using a PHERAstar FS microplate reader (BMG Labtech). The TR-FRET signal of each data point was extracted by calculating the 520/490 nm ratio.

### ELISA Assay for Spike Protein

The ELISA Assay was conducted in 384-well plate (Thermo Fisher #464718), which was coated with 50 μL/well of 500 ng/mL SARS-CoV-2 S protein in coating buffer (1 capsule of carbonate-bicarbonate buffer (Sigma #C3041100CAP) per 100 mL Milli-Q H₂O) for 30 minutes at room temperature. The plates were then washed 3 times with 100 μL/well of wash buffer (0.05% Tween-20, 400 mM NaCl, 50 mM Tris pH 8.0 in Milli-Q H₂O) using a Tecan automated plate washer. The plates were blocked by adding 100 μL/well of blocking buffer (1% BSA, 140 mM NaCl, 50 mM Tris pH 8.0 in Milli-Q H₂O) for 30 minutes at room temperature. The plates were then washed as described above. Samples were diluted to a volume of 50 μL (in dilution buffer; 1% BSA, 0.05% Tween-20, 140 mM NaCl, 50 mM Tris (pH 8.0) in Milli-Q H₂O) prior to addition to the wells and were incubated for 30 minutes at 37° C. The plates were then washed 5 times as described above. Detection antibody solution was diluted to a volume of 50 μL/well (HRP-anti human IgG Bethyl Laboratory #A80-104P) prior to addition to the wells and was incubated for 30 minutes at room temperature. Plates were then washed 5 times as described above. TMB peroxidase substrate (40 μL/well; Thermo Fisher #34029) was then added to the wells and incubated at room temperature for 3 minutes (IgG). The reaction was stopped by adding 40 μL/well of stop solution (1 M H₂SO₄ in Milli-Q H₂O) to each well. OD were read at 450 nm and 570 nm on a Pherastar FSX plate reader. The final data used in the analysis was calculated by subtracting 570 nm background from 450 nm signal.

### ELISA Assay for N Protein

The ELISA Assay was conducted in 384-well plate (Thermo Fisher #464718), which was coated with 50 μL/well of 500 ng/mL SARS-CoV-2 N protein in coating buffer (1 capsule of carbonate-bicarbonate buffer (Sigma #C3041100CAP) per 100 mL Milli-Q H₂O) for 30 minutes at room temperature. The plates were then washed 3 times with 100 μL/well of wash buffer (0.05% Tween-20, 400 mM NaCl, 50 mM Tris pH 8.0 in Milli-Q H₂O) using a Tecan automated plate washer. The plates were blocked by adding 100 μL/well of blocking buffer (1% BSA, 140 mM NaCl, 50 mM Tris pH 8.0 in Milli-Q H₂O) for 30 minutes at room temperature. The plates were then washed as described above. Samples were diluted to a volume of 50 μL (in dilution buffer; 1% BSA, 0.05% Tween-20, 140 mM NaCl, 50 mM Tris (pH 8.0) in Milli-Q H₂O) prior to addition to the wells and were incubated for 30 minutes at 37° C. The plates were then washed 5 times as described above. Detection antibody solution was diluted to a volume of 50 μL/well (HRP-anti human IgG Bethyl Laboratory #A80-104P) prior to addition to the wells and was incubated for 30 minutes at room temperature. Plates were then washed 5 times as described above. TMB peroxidase substrate (40 μL/well; Thermo Fisher #34029) was then added to the wells and incubated at room temperature for 3 minutes (IgG). The reaction was stopped by adding 40 μL/well of stop solution (1 M H₂SO₄ in Milli-Q H₂O) to each well. OD were read at 450 nm and 570 nm on a Pherastar FSX plate reader. The final data used in the analysis was calculated by subtracting 570 nm background from 450 nm signal.

### Statistics

Statistical calculations were performed using Prism 8.0.2 and R v3.6.1; packages ggplot2. The correlation plots include geometrical smoothing using R v3.6.1 geom_smooth function with generalized linear model calculated (glm method) confidence intervals. The samples in ELISA IgG or TR-FRET IgG was classified as positive if the value exceeded the mean (healthy)+3 standard deviation (healthy) threshold.

### Example 2: Development of a TR-FRET Assay to Detect SARS-CoV-2 Antibodies

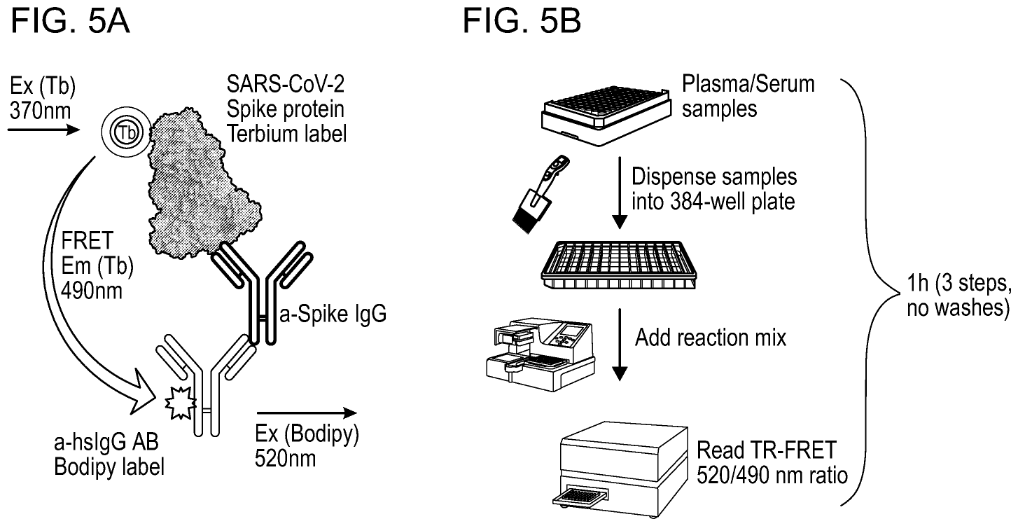

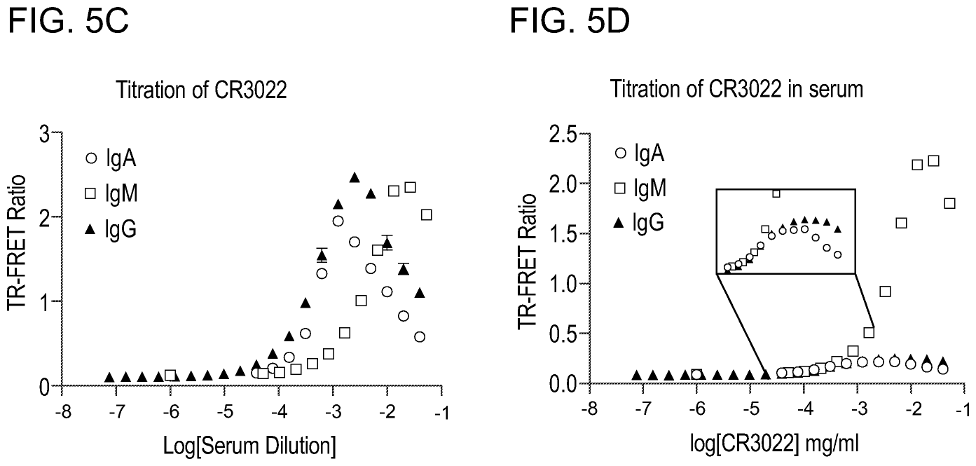

A homogenous serological assay was developed for the detection of SARS-CoV-2 antibodies in human plasma/serum that is based on TR-FRET detection (FIG. 5A). The assay allows for a simple mix-and-read protocol that easily lends itself to scalable automation (FIG. 5B). The assay is based on the detection of a ternary complex comprising a donor fluorophore labeled antigen and an acceptor fluorophore labeled detection antibody, with recognition initiated by the serum immunoglobulins (FIG. 5A). The proximity of the donor (Terbium) and acceptor (BODIPY) fluorophores results in a positive FRET signal that is read out as a 520 nm (acceptor)/490 nm (donor) ratio and allows for accurate quantification of the serum antibodies in an immunoglobulin isotype specific manner.

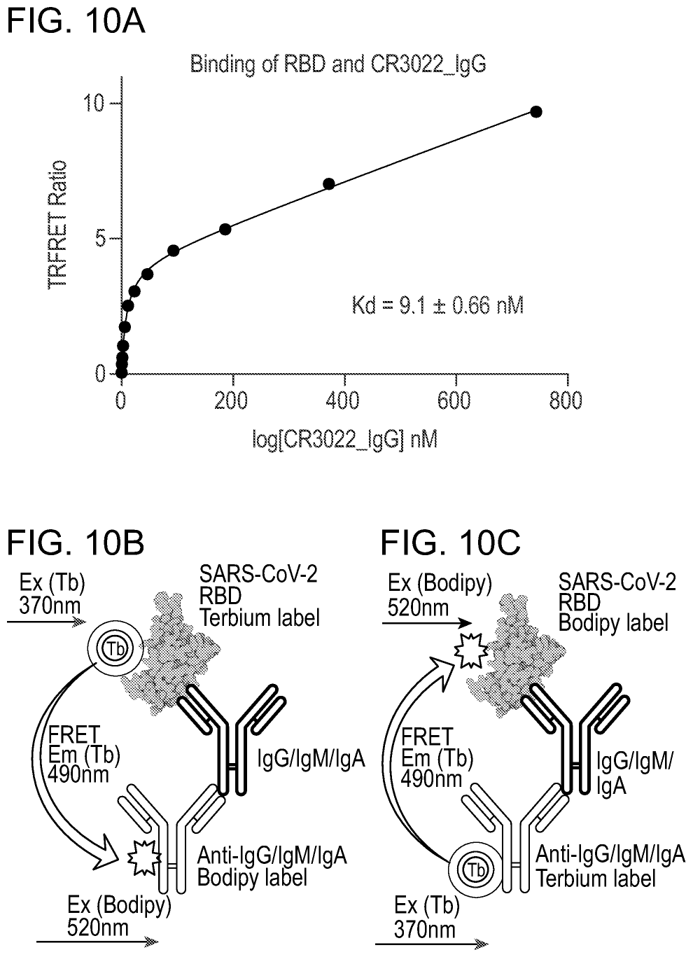

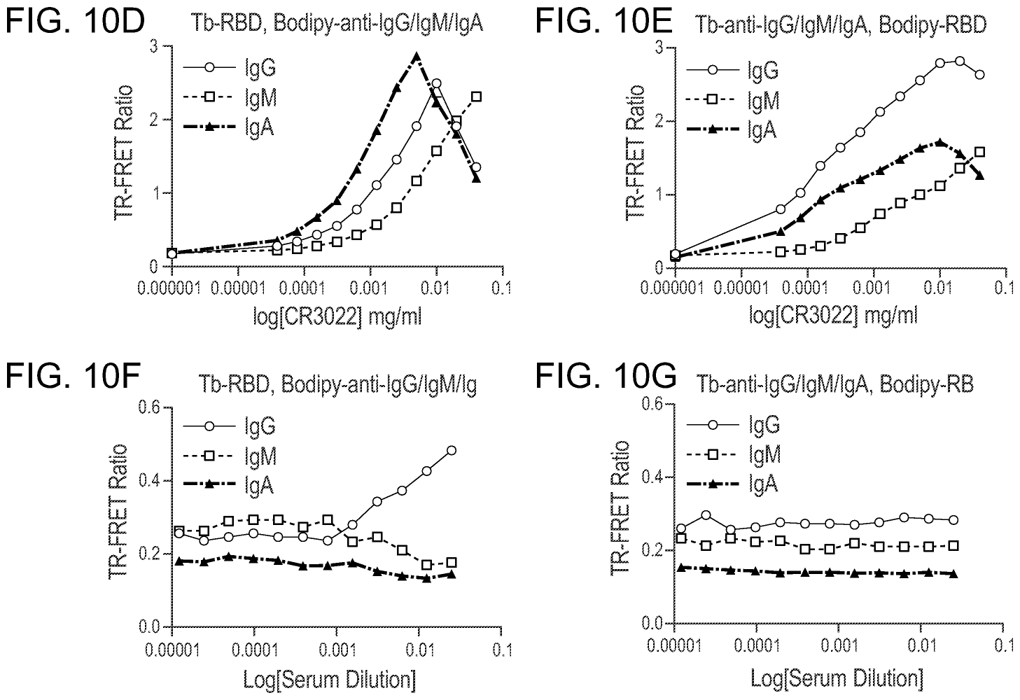

To enable sensitive detection, minimization of background signal with simultaneous optimization of specific signal is critical especially since the homogenous assay format lacks any signal amplification. First, it was established that the TR-FRET assay format can detect the binding of immunoglobulin variants IgG, IgM and IgA1 to SARS-CoV-2 antigens (FIG. 5C). The receptor binding domain (RBD) of SARS-CoV-2 spike protein was recombinantly expressed and labeled with Terbium-NETS or BODIPY-NHS detection antibodies (anti-IgG, anti-IgM, anti-IgA1), which were commercially obtained and also labeled with either Terbium-NHS or BODIPY-NHS. The SARS-1 IgG antibody CR3022 was recombinantly expressed and cross-reacted with the RBD of SARS-CoV-2 (Kd of 9.1±0.66 nM, FIG. 10A) along with IgM and IgA1 containing the CR3022 variable region (Tian et al., 2020 Emerg. Microbes Infect., 9:382-385).

Titrations of CR3022 (IgG, IgM, IgA1) were performed by adding a mix of labeled RBD and labeled detection antibody to vary the position of the donor and acceptor fluorophore (either on RBD or detection antibody) (FIG. 10B, 10C). While all combinations led to a functional readout (FIG. 10D, 10E), the detection antibody signal was diluted across the entire immunoglobulin pool when performed in serum or plasma samples, which lead to signal loss (FIG. 10F, 10G). For optimization, the antigen was labeled with terbium and the detection antibody labeled with BODIPY, resulting in quantitative binding curves for IgG/IgM/IgA1 (FIG. 5C). The binding curves exhibit the characteristic bell-shape due to the prozone effect (Ha et al., 2016 Cell Rep., 16:2047), which can be accurately accounted for by mathematical models (Douglass et al., 2013 J Am Chem Soc, 135:6092-6099).

Next, it was established that CR3022 can similarly be detected in human serum (FIG. 5D). While the signal was drastically reduced, the low background level allowed for accurate quantification. During the optimization of assay conditions, replacing RBD with the full-length SARS-CoV-2 spike protein (S protein) significantly reduced background specifically in presence of serum. This was likely due to 1) the trimeric nature of the full S protein leading to avidity effects, and 2) the RBD exposing surfaces that are shielded in the full-length S protein and result in unspecific interactions.

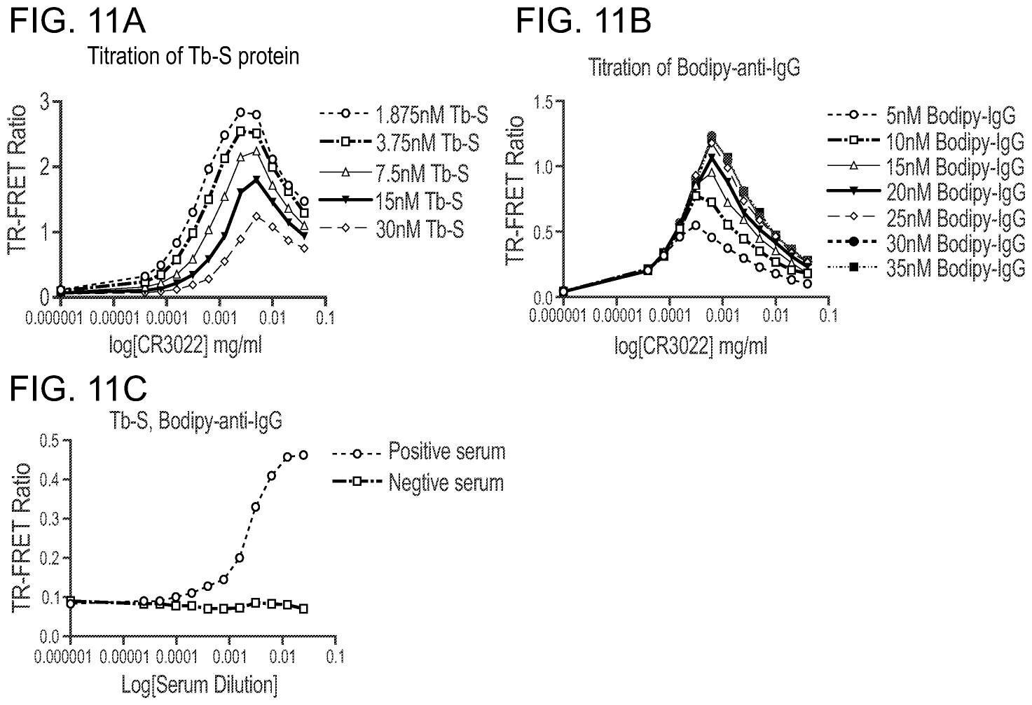

Further validation was continued using Spike protein, and subsequently the concentrations of antigen and detection antibody were optimized (FIG. 11A, 11B). With optimized concentrations, it was validated that convalescent serum results in a dose dependent response in TR-FRET signal (FIG. 11C).

### Example 3: Homogenous TR-FRET Assay Can Detect IgG in Patient Serum

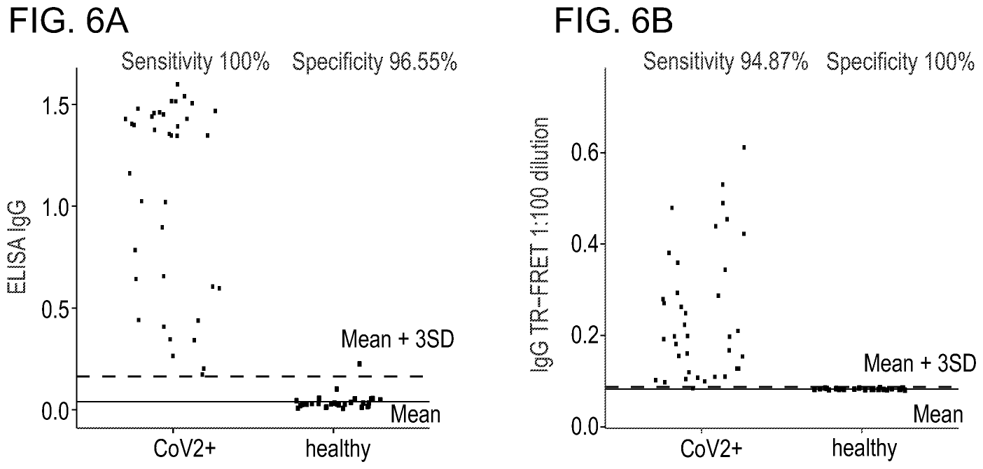

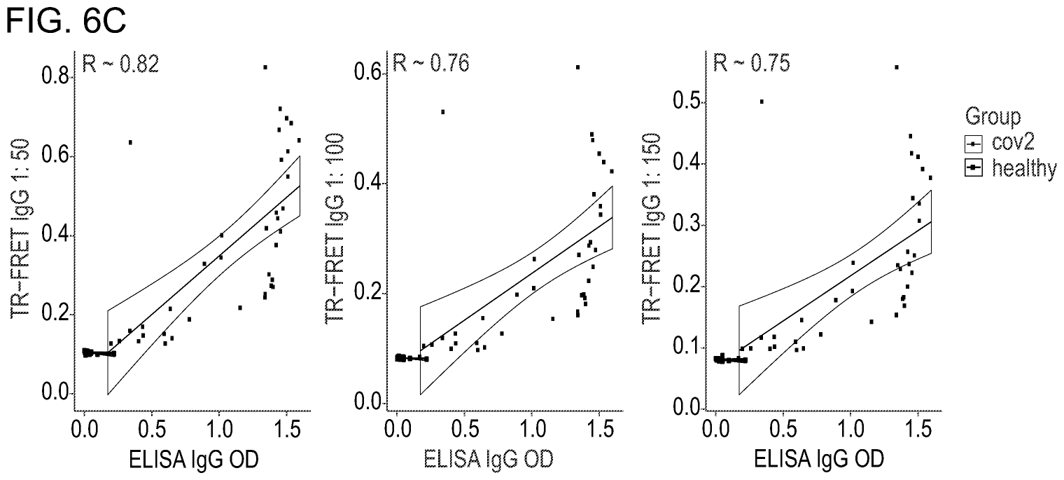

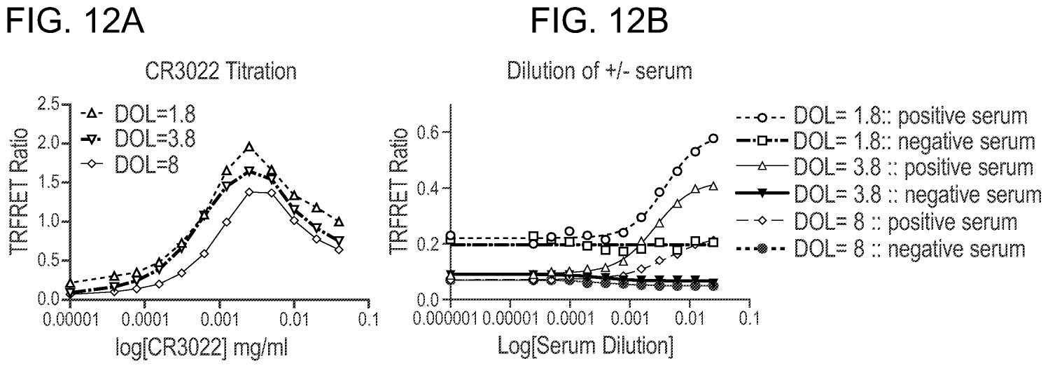

After optimizing the assay conditions, the detection of antibodies in serum obtained from convalescent patients (CoV2+) and pre-pandemic negative control serum (healthy) was tested. A set of 49 PCR tested positive and 28 PCR tested negative serum samples was assembled (hereafter referred to as 96w_testset). An ELISA using Spike protein was performed as a reference (FIG. 6A). The TR-FRET assay was performed on the 96w_testset at an initial serum dilution of 1:100 to match the exact ELISA concentration (FIG. 6B). The TR-FRET achieved 94.87% sensitivity and 100% specificity, which was comparable to the ELISA that achieved 100% sensitivity and 96.55% specificity, when measured with a cutoff based on 3 standard deviations away from the healthy control mean. A strong correlation between the TR-FRET and ELISA assays (FIG. 11C) was observed. While the discrimination between CoV2+ and CoV2− was comparable between TR-FRET and ELISA, the ELISA had significantly stronger signal compared to TR-FRET especially for low responders. This was most likely due to the facts that the ELISA was a signal amplification assay that was compared to the equilibrium binding of the TR-FRET and that the ELISA was offset by the low background noise of the TR-FRET. The assay was performed again with the 96w_testset using dilution factors of 1:150, 1:100, and 1:50. It was observed that increasing the serum concentration improves performances without compromising background noise (FIG. 6C, 6D). All concentrations had equivalent or better discrimination between Cov2+ and negative serum when compared to the ELISA (FIG. 6). Since the TR-FRET assay utilized covalent labeling of the antigen with terbium, the Degree of Labelling (DOL) was optimized to ensure that no epitope masking occurred (FIG. 12A-12B). A DOL equivalent to approximately 3.8 provided no detectable epitope masking with optimal signal. A DOL equivalent to approximately 3.8 was used for all additional experiments.

### Example 4: The TR-FRET Assay Can Accurately Detect Seroconversion

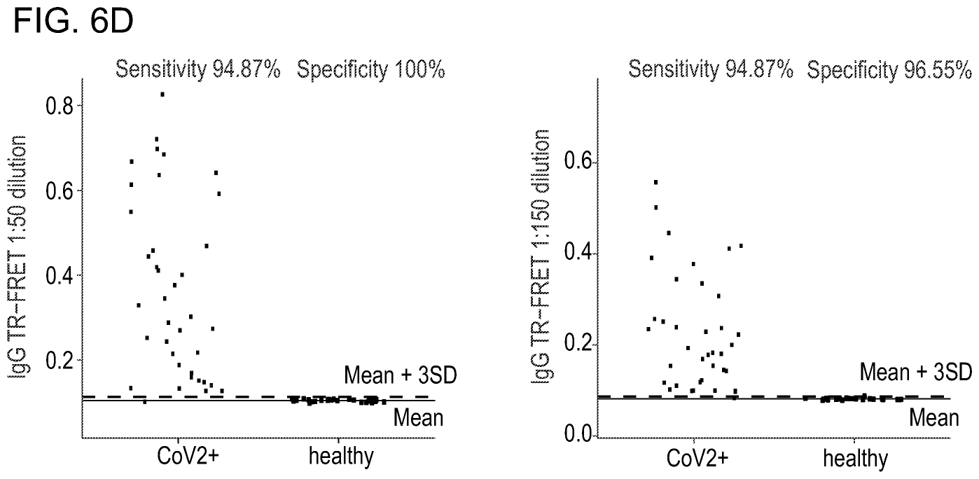

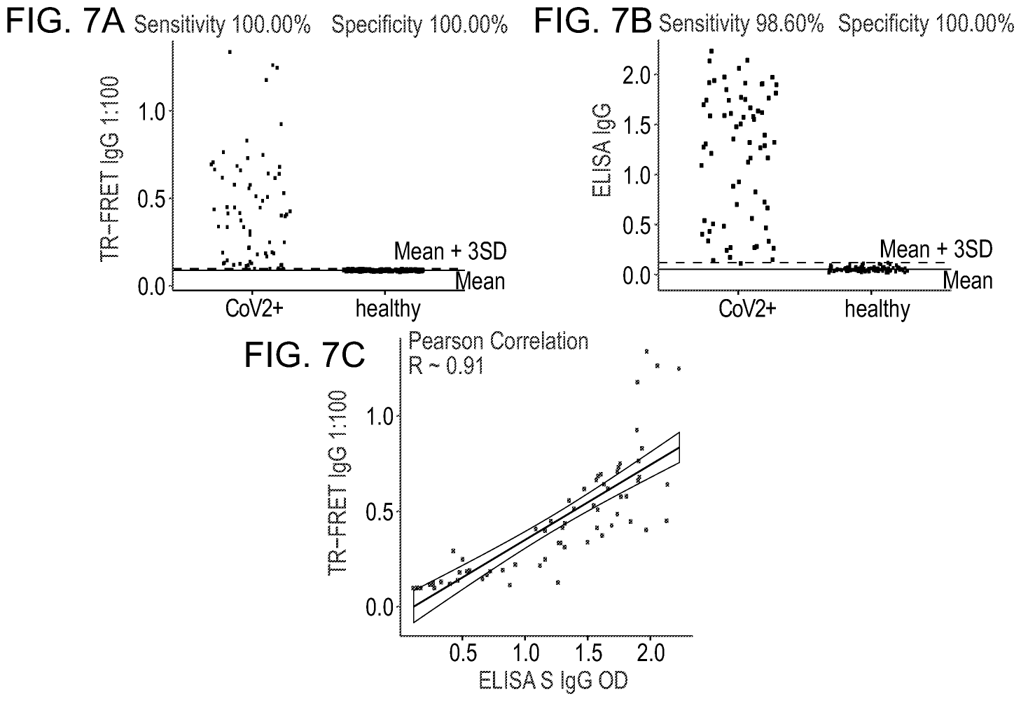

The TR-FRET assay was used to detect seroconversion in a larger set of samples containing 68 SARS-CoV-2 PCR positive samples (CoV2+), and 100 pre-pandemic negative samples (Healthy) (hereafter referred to as BWH set). These samples were also profiled using the established ELISA assay. In line with previous observations, the standard deviation of the healthy controls was very low, and accurate discrimination between CoV2+ and healthy samples was achieved with 100% specificity and 100% sensitivity when using a cut-off based on 3 standard deviations of the healthy control (FIG. 7A). For direct comparison, the established ELISA assay was performed on the same sample set (FIG. 7B), which gave comparable results. The response of individual samples between TR-FRET and ELISA assays was well correlated (FIG. 7C).

### Example 5: The Analytical Limit of Detection (LoD) for the TR-FRET Assay

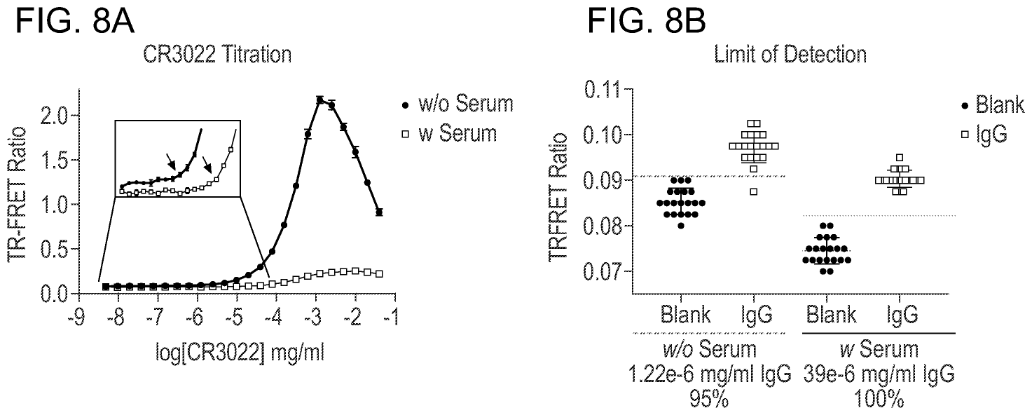

In order to assess the limit of detection (LoD) of the TR-FRET assay, a titration of control antibody CR3022 IgG was performed in the presence and absence of negative control serum at 1:150 serum to buffer dilution (FIG. 8A). The prozone effect was clearly visible at higher concentrations of the antibody and signal intensity was reduced in the presence of serum. The lowest concentrations of CR3022 antibody where signal was higher than mean+3 s.d. was selected in order to establish LoD. Comparison of 20 replicates was performed against 20 replicates of blank control in presence and absence of serum (FIG. 8B). Based on this, the LoD for the TR-FRET assay was determined to be 1.22 ng/mL in the absence of serum and 39 ng/mL in presence of the serum, which was comparable to the 32 ng/mL 21 LoD for the ELISA assay.

### Example 6: Assessing Intra- and Inter-Assay Precision for the TF-FRET Assay

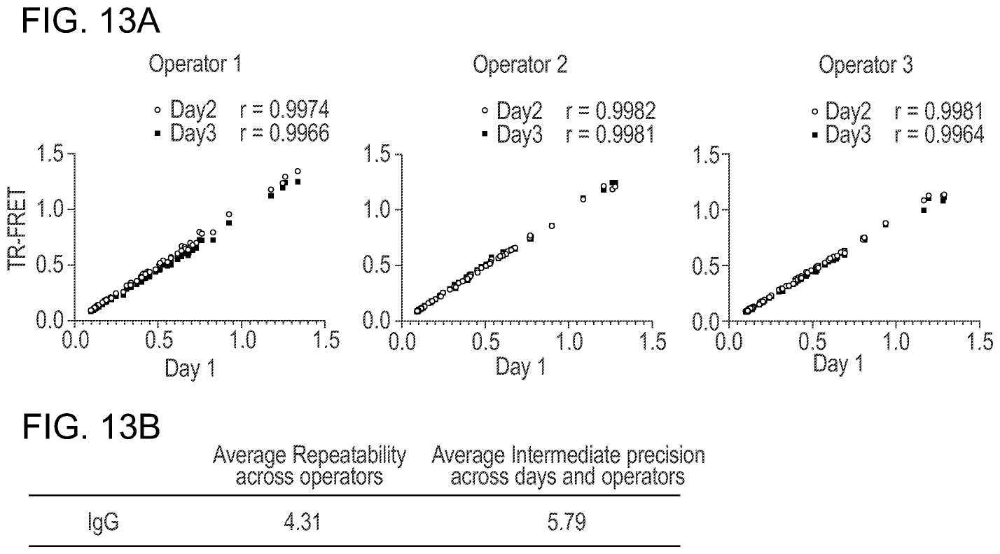

Eliminating the wash steps and reducing the overall number of sample handling steps should produce high reproducibility and repeatability. To assess the intra- and inter-assay precision of the TR-FRET assay, a set of positive responders as well as negative control samples (68 total) were selected. The assay was performed with three operators on three different days (FIG. 13A-13B). The correlation between operators was above 99.6% with average repeatability of 4.31% and overall precision across days and operators of 5.72%, which is well within the desired range.

### Example 7: The TR-FRET Assay Can be Rapidly Extended to Additional SARS-CoV-2 Antigens

After establishing a serological assay for Spike protein, it was assessed whether the TR-FRET setup was compatible with other antigens. Spike protein is one of the most widely studied antigens in serological assays for SARS-CoV-2, but there are other SARS-CoV-2 proteins that are highly immunogenic (Dutta et al., 2020 J Virol., 94), such as the highly abundant nucleocapsid protein (N protein) that binds to viral RNA inside the virion (Lu et al., 2020 Lancet, 395:565-574; Narayanan et al., 2003 J Virol, 77:2922-2927).

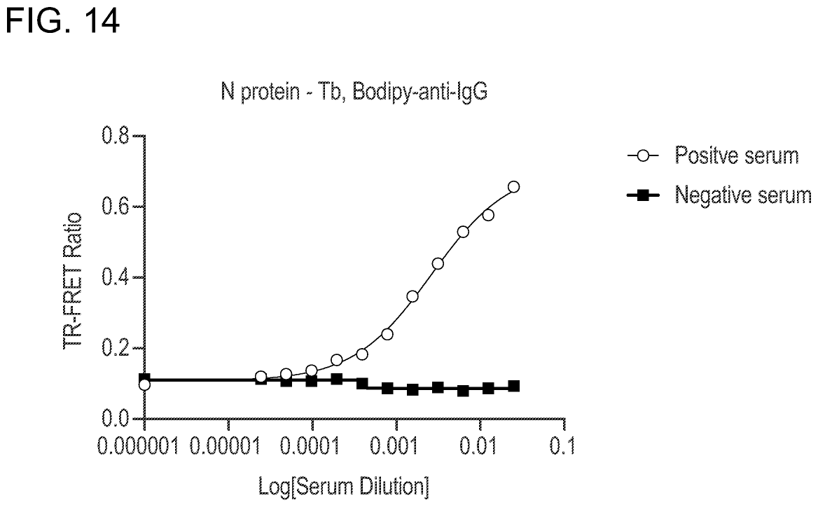

An N protein TR-FRET IgG detection assay (thereafter named N TR-FRET) was established. The same TR-FRET setup was used, where the donor fluorophore was on the antigen and the acceptor fluorophore was on the IgG antibody. A commercial Biotinylated N protein was obtained and labeled with a terbium-streptavidin conjugate. A titration of convalescent CoV2+ serum into biotinylated N protein, Tb-Streptavidin, and BODIPY-IgG was performed to validate the assay. A dose response was observed with strong signal present at a lower dilution (1:50), which was consistent with our Spike protein TR-FRET assay (FIG. 14).

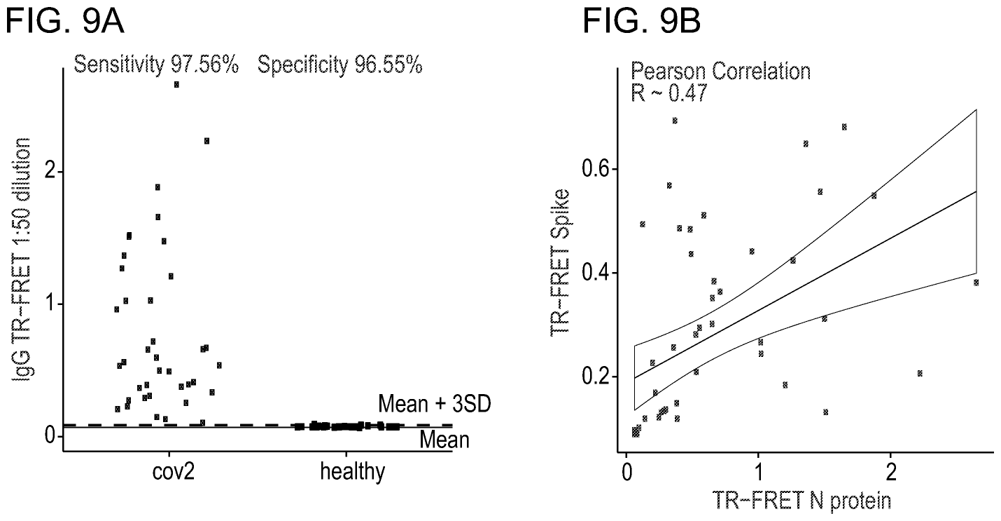

N TR-FRET was performed on the 96w_testset, resulting in a sensitivity of 97.56% and specificity 96.55% (FIG. 9A). Interestingly, the TR-FRET signal strength of N TR-FRET exceeded that of the S TR-FRET assay (FIG. 9B). The Spike TR-FRET and N TR-FRET readouts on the 96w_testset were compared, resulting in a Pearson Correlation coefficient of 0.47. This indicated that the two assays were highly orthogonal and were likely to provide additive information on serological status when combined (FIG. 9B).

### Example 8: TR-FRET Assay Using Dried Whole Blood Samples

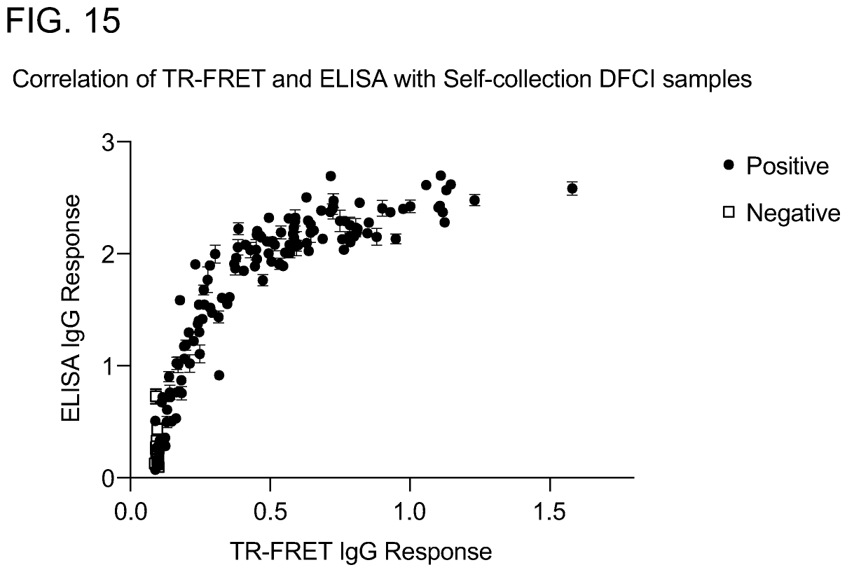

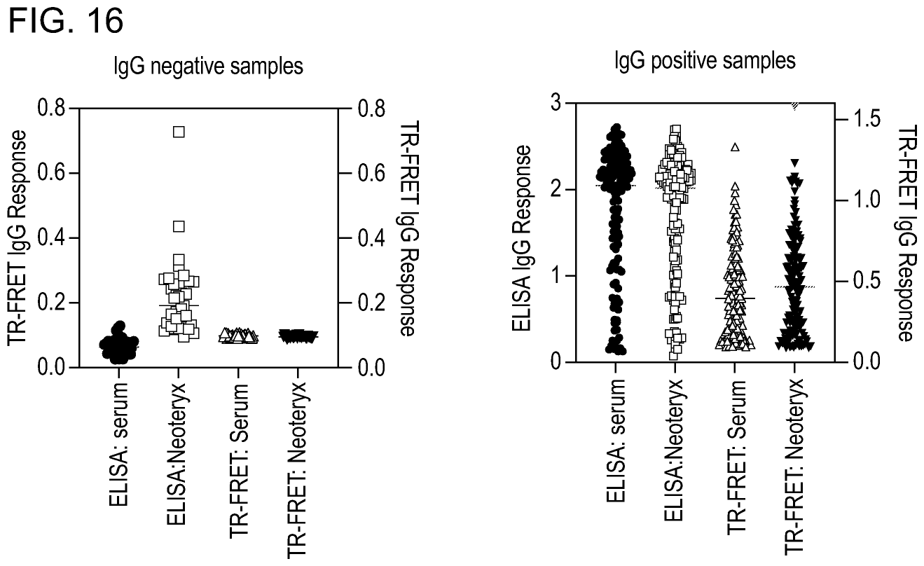

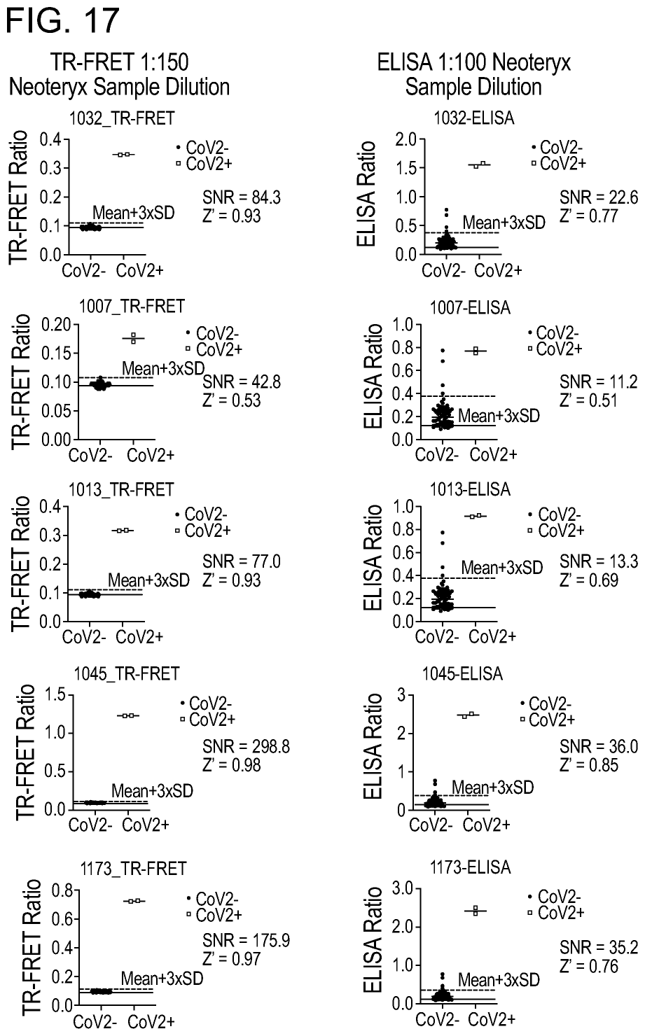

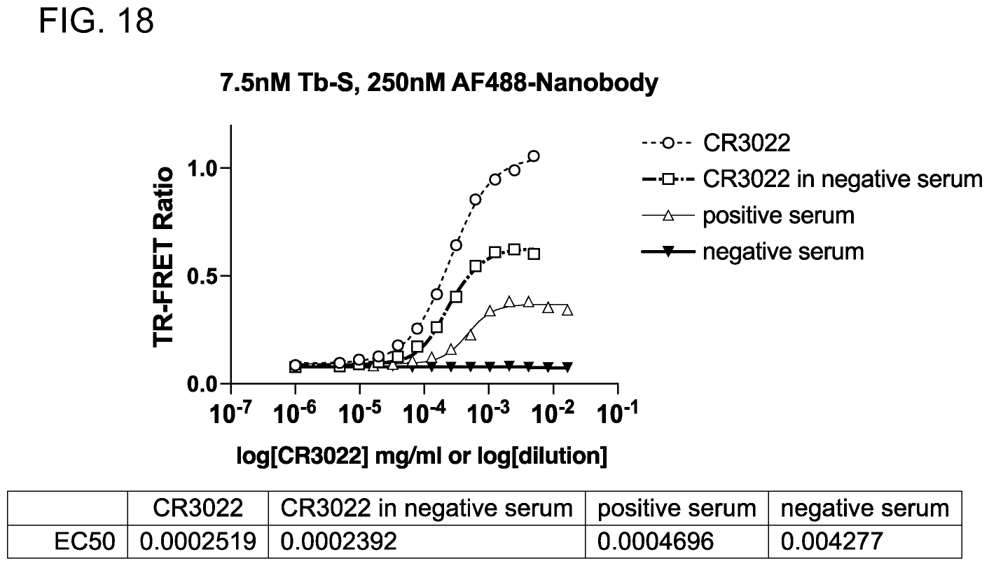

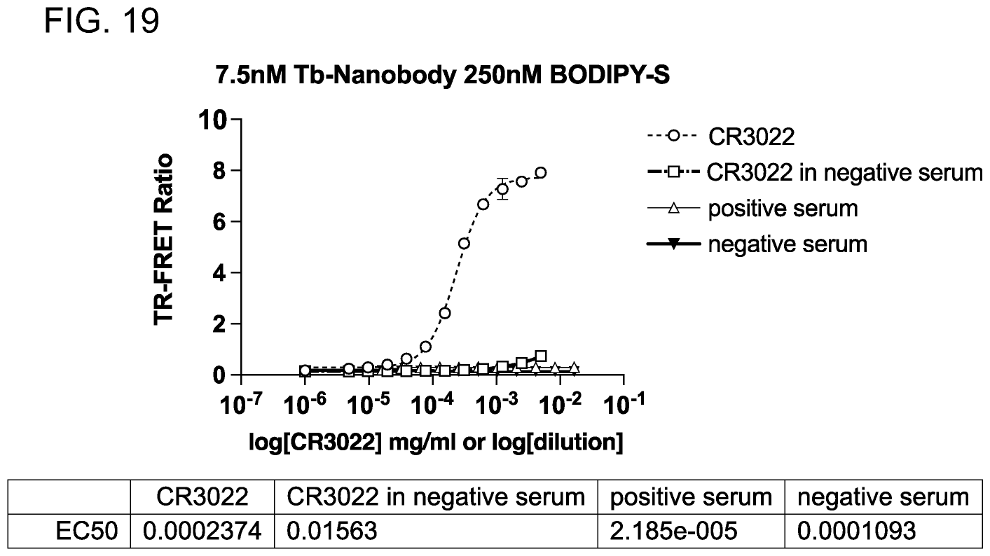

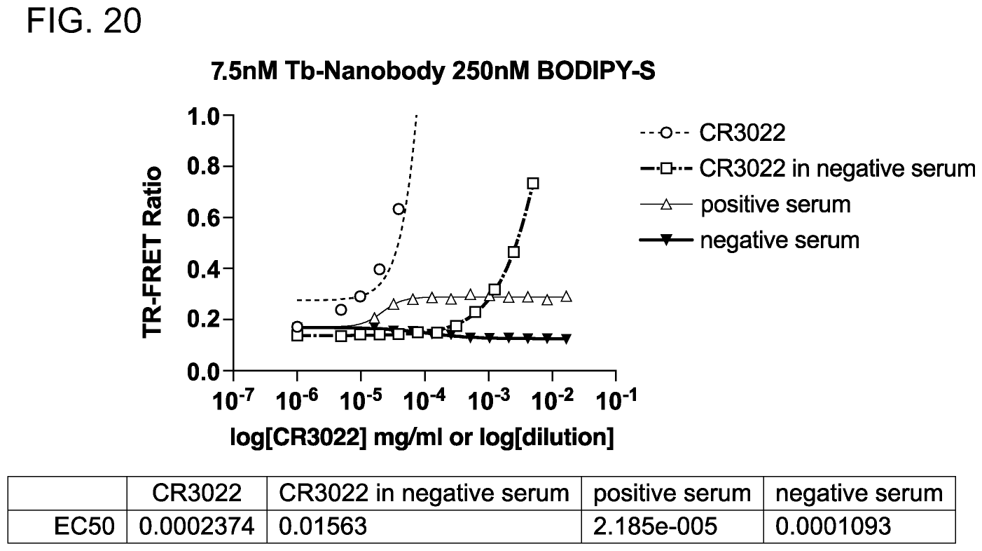

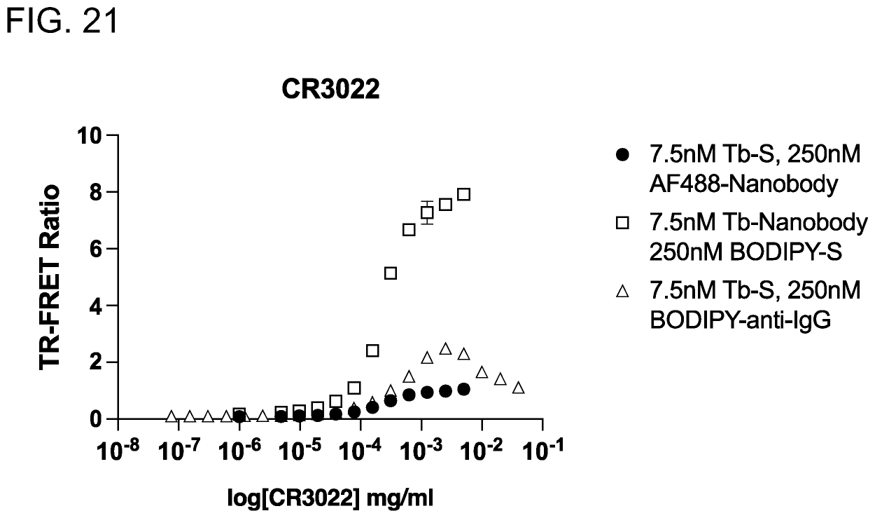

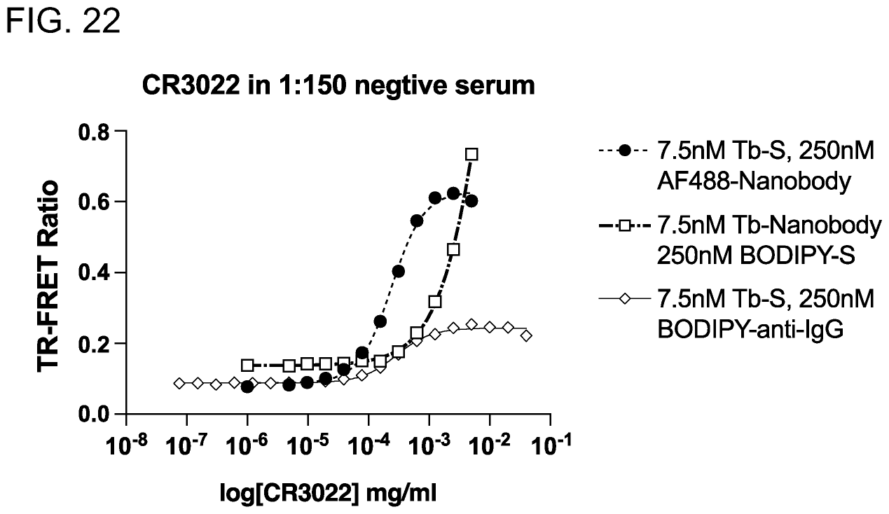

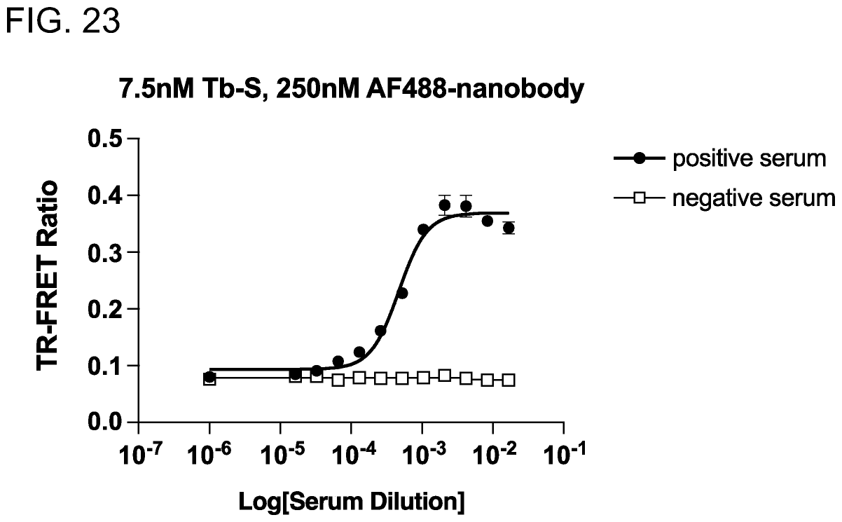

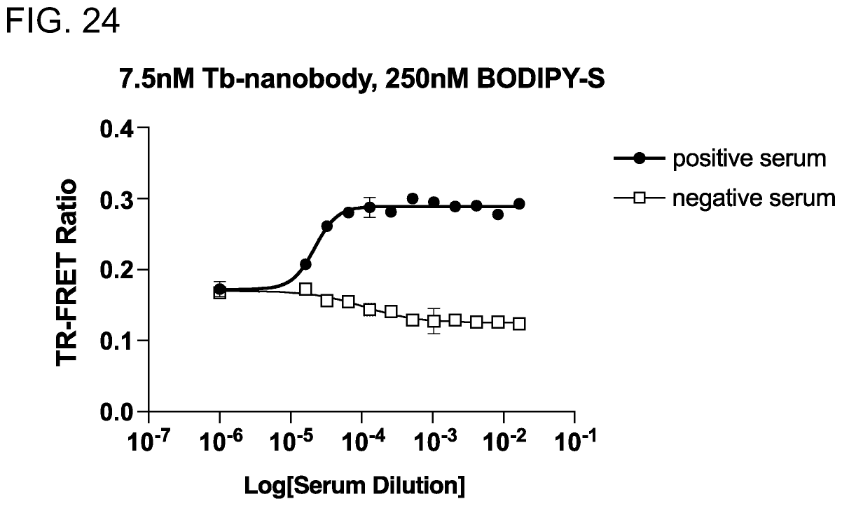

The robustness of the TR-FRET IgG-S assay was demonstrated using dried whole blood samples (N=175). Low variability of the background signal across serum and whole blood sample types (Neoteryx) was observed for the TR-FRET results, a hallmark of the TR-FRET assay, which was not the case for ELISA measurements, where background significantly increased in variability for the whole blood sample (FIG. 15-FIG. 17). These results led to reduced signal to noise and Z′ in the ELISA assay, while the performance of the TR-FRET assay was not altered (FIG. 17).

### Example 9: TR-FRET Assay Can Rapidly be Extended to Additional Antigens

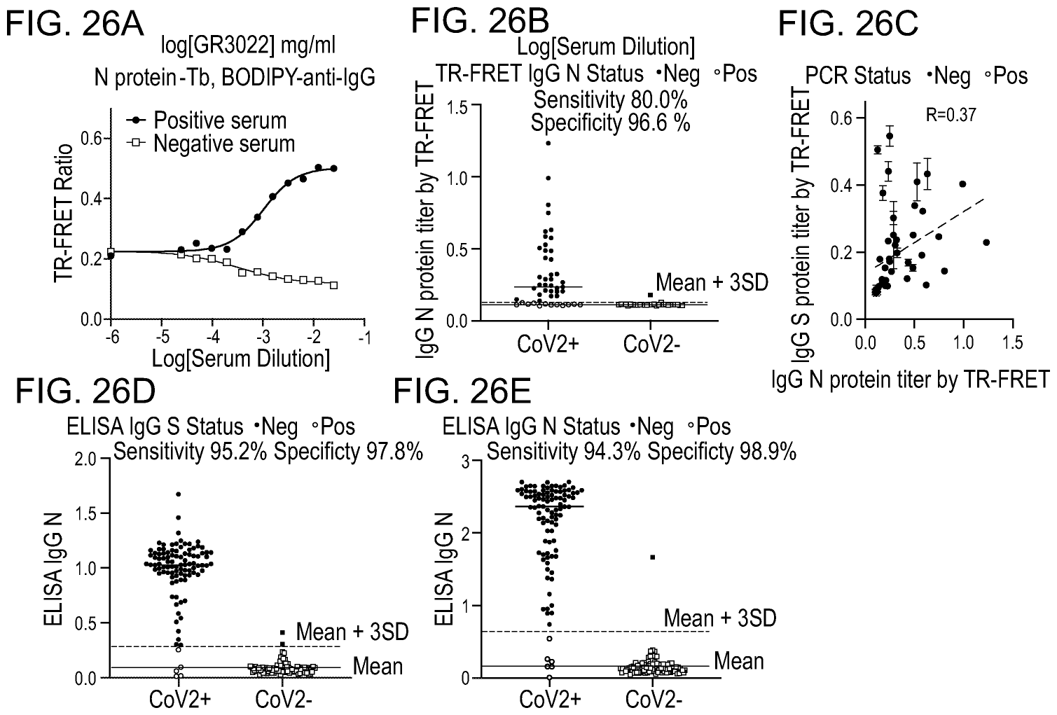

Having an established serological assay for S protein, whether the TR-FRET setup was compatible with other antigens could be determined. S protein or S-RBD are the most widely used antigens in serological assays for SARS-CoV-2, but there are other SARS-CoV-2 proteins that are highly immunogenic, such as the abundant nucleocapsid protein (N protein), which binds to viral RNA inside the virion. An N protein TR-FRET IgG detection assay was established (thereafter named N TR-FRET) utilizing the same TR-FRET setup as before, with the donor fluorophore on the antigen and the acceptor fluorophore on the αIgG antibody. The N protein was expressed from insect cells, biotinylated and terbium-streptavidin (Tb-SA) conjugate was used to label the antigen. To validate the assay setup, a titration of convalescent CoV2+ serum into biotinylated N protein was performed, Tb-SA and BODIPY-αIgG. A dose response with strong signal present at dilution of 1:150 was observed, consistent with the S-protein TR-FRET (FIG. 26A). Next, N TR-FRET was performed on the 96w_testset which resulted in a sensitivity of 80.0% and specificity 96.6% (FIG. 26B).

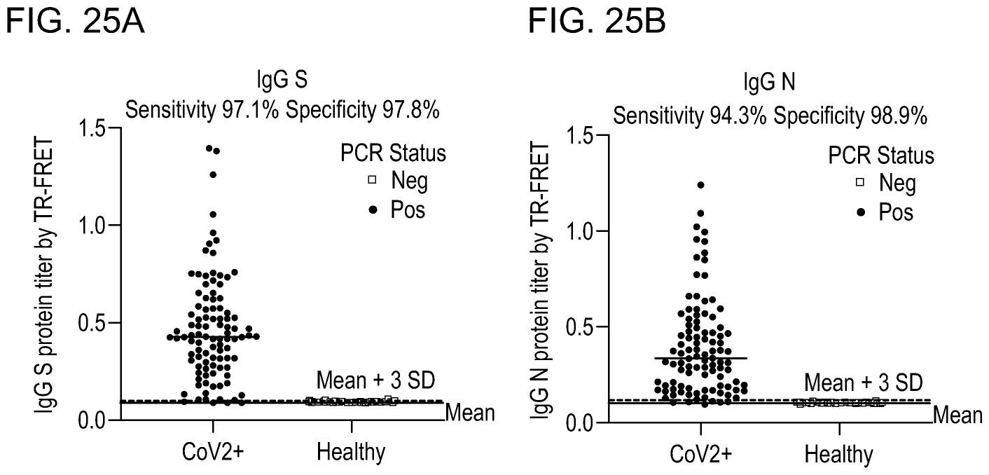

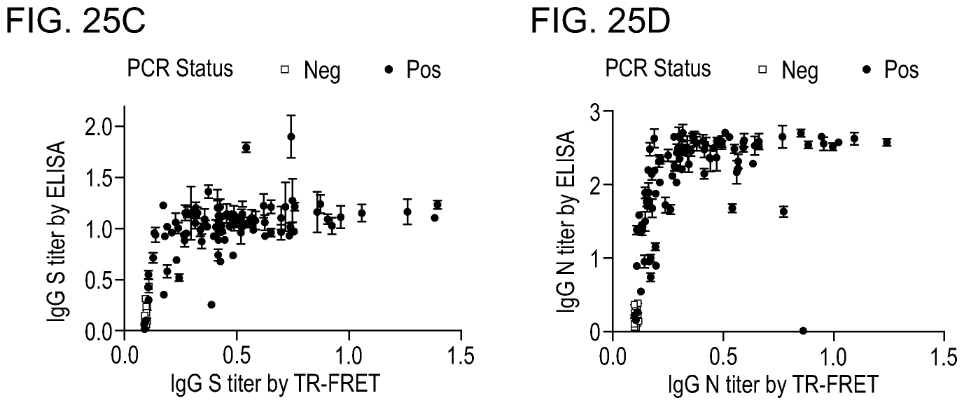

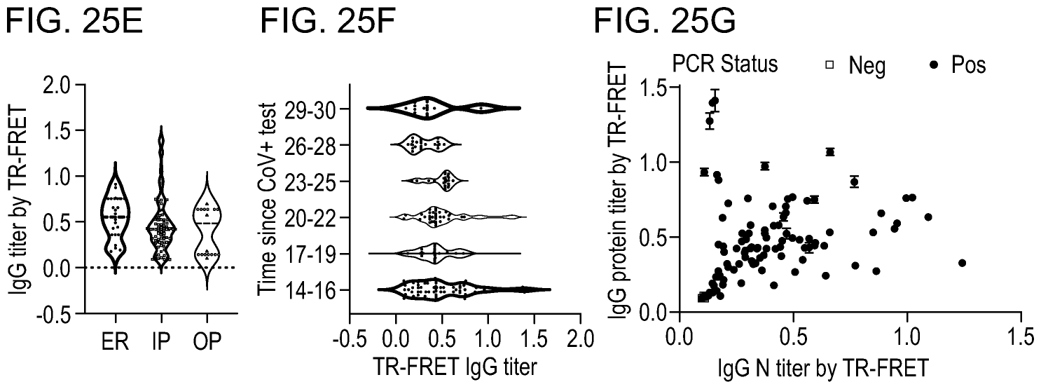

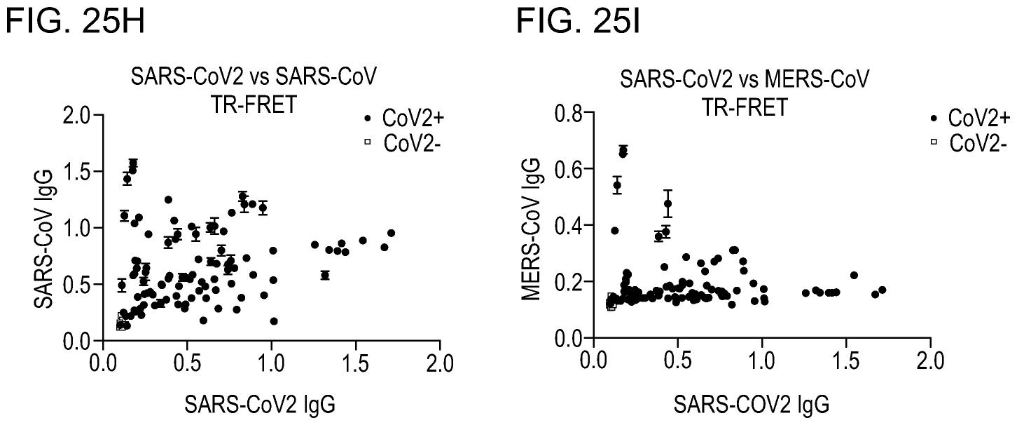

In order to further assess performance of the established IgG S and N TR-FRET assays, a larger sample set from the Mass CPR consortium was utilized with 100 SARS-CoV-2 RT-PCR positive samples (CoV2+), as well as 90 pre-pandemic controls from the Dana-Faber Cancer Institute Bio Bank (heathy, CoV2−), thereafter named MassCPR set. Using the established mean (healthy)+3 SD (healthy) cutoff, the TR-FRET assay performance was established with 97.1% sensitivity and 97.8% specificity, respectively, for the S antigen and 95.2% sensitivity and 98.9% specificity for the N antigen (FIG. 25A and FIG. 25B). The analogous results using the ELISA resulted in 95.2% sensitivity and 97.8% specificity for the S antigen and 94.3% sensitivity and 98.9% specificity for the N antigen (FIG. 26D and FIG. 26E). In both S and N assays TR-FRET showed improved sensitivity over ELISA (97.1% for S TR-FRET, 95.2% for S ELISA, and 95.2% for N TR-FRET, 94.3% for N ELISA) with identical specificity. As seen before with the MGB set, a ‘ceiling’ of signal was noticed with the ELISA readout and increased dynamic range for the TR-FRET assay (FIG. 25C and FIG. 25D). The clinical admission status of the MassCPR sample cohort indicated 19 patients were admitted to the emergency room (ER), 76 as inpatients (IP) and 5 as outpatients (OP). A significant difference in the IgG S antibody titers between the groups was not observed (FIG. 25E). The number of days since the last positive SARS-CoV-2 test was recorded and within the 14-30-day period IgG levels varied without significant trends (FIG. 25F), in line with previously reported longitudinal studies where the S IgG level stabilizes 14 days post infection. Comparing the S and N TR-FRET readouts on the MassCPR and 96w_testset resulted in a Pearson correlation coefficient of 0.37 for 96w_testset (FIG. 26C) and 0.22 for MassCPR (FIG. 25G) indicating that the two assays were partially orthogonal and likely provide additive information on serological status when combined, which is in accordance with what has been found in other studies (FIG. 25G and FIG. 26C). The spike protein has high sequence similarity between SARS-CoV-2 and SARS-CoV and to lesser extend MERS-CoV which can result in cross reactivity in the antibody response. In analogous fashion to the SARS-CoV-2 S TR-FRET assay, S based IgG detection assays for SARS-CoV and MERS-CoV were established and tested using the MassCPR set of samples. As expected, cross-reactivity between SARS-CoV-2 and SARS-CoV was observed (FIG. 25H), but very limited cross-reactivity with MERS-CoV (FIG. 25I). This again was in line with previous observations using these antigens and demonstrates that the TR-FRET assay behaves similar to ELISA and other formats and that cross-reactivity or sensitivity are largely determined by the choice of antigen. Samples with high titer of IgG antibodies against MERS-CoV S have been identified in ˜6% of CoV2+ samples tested.

All patent publications and non-patent publications are indicative of the level of skill of those skilled in the art to which this invention pertains.

All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A homogeneous, TR-FRET-based method for detection of an anti-betacoronavirus (β-CoV) antibody in a patient fluid sample, comprising obtaining a body fluid sample from a patient; contacting the body fluid sample with a first reagent and a second reagent, thus forming an assay mixture, wherein each of the first and second reagents binds the β-CoV antibody (the primary antibody), wherein the first reagent comprises a first subpopulation of a β-CoV antigen and the second reagent comprises a second subpopulation of the β-CoV antigen, wherein the first and second subpopulations of the β-CoV antigen are differentially labeled with a donor fluorophore and an acceptor fluorophore, or wherein the first reagent comprises the β-CoV antigen and the second reagent comprises a secondary antibody or a nanobody that binds the primary antibody, and wherein the first and second reagents are differentially labeled with a donor fluorophore and an acceptor fluorophore; and detecting a FRET signal, wherein detection of a FRET signal indicates presence of the first reagent comprising the first subpopulation of a β-CoV antibodies in the body fluid sample and is diagnostic of an infection with a β-CoV.

2. The method of claim 1, wherein the body fluid sample comprises whole blood, serum or plasma.

3. The method of claim 1, wherein the donor fluorophore is a lanthanide metal, or a complex thereof.

4. The method of claim 3, wherein the lanthanide metal is terbium (Tb) or europium (Eu), or wherein the donor fluorophore is a chelate or cryptate of terbium or europium.

5. (canceled)

6. The method of claim 4, wherein the donor fluorophore is terbium.

7. The method of claim 1, wherein the acceptor fluorophore is selected from the group consisting of organoboron fluorescent dyes, allophycocyanins, rhodamines, cyanines, squaraines, coumarins, proflavins, acridines, fluoresceins, boron-dipyrromethene derivatives, sodium 6-amino-9-(5-((aminomethyl)carbamoyl)-2-carboxyphenyl)-3-iminio-3H-xanthene-4,5-disulfonate, 2-[5-[3,3-dimethyl-5-sulfo-1-(3-sulfopropyl)indol-1-ium-2-yl]penta-2,4-dienylidene]-3-methyl-3-[5-oxo-5-(6-phosphonooxyhexylamino)pentyl]-1-(3-sulfopropyl)indole-5-sulfonic acid (ALEXA647), and nitrobenzoxadiazole.

8. The method of claim 7, wherein the acceptor fluorophore is an organoboron dye.

9. The method of claim 8, wherein the organoboron dye is boron-dipyrromethene (BODIPY).

10. The method of claim 1, wherein the β-CoV antigen comprises a β-CoV Spike Protein or an antigenic fragment thereof, or wherein the β-CoV antigen comprises a β-CoV nucleocapsid protein (N protein), or an antigenic fragment thereof.

11. The method of claim 10, wherein the Spike Protein comprises subunit 1 of Spike protein (S1), subunit 2 of Spike protein (S2), or the receptor binding domain (S1-RBD).

12. (canceled)

13. The method of claim 1, wherein the first reagent comprises the first subpopulation of a β-CoV antigen and the second reagent comprises the second subpopulation of the β-CoV antigen, wherein the first and second subpopulations of the β-CoV antigen are differentially labeled with a donor fluorophore and an acceptor fluorophore.

14. The method of claim 1, wherein the first reagent comprises the β-CoV antigen, and the second reagent comprises a secondary antibody or a nanobody that binds the primary antibody, and wherein the first and second reagents are differentially labeled with a donor fluorophore and an acceptor fluorophore.

15. The method of claim 1, wherein the first reagent contains the donor fluorophore, and the second reagent contains the acceptor fluorophore.

16. The method of claim 1, wherein the first reagent contains the acceptor fluorophore, and the second reagent contains the donor fluorophore.

17. The method of claim 14, wherein the second reagent binds a specific class of human antibodies or wherein the second reagent comprises an antibody or nanobody that binds a specific subtype of human antibody.

18. The method of claim 17, wherein the second reagent comprises an anti-IgG antibody or an anti-IgG nanobody, or wherein the second reagent comprises an anti-IgM antibody or an anti-IgM nanobody, or wherein the second reagent comprises an anti-IgA antibody or an anti-IgA nanobody.

19.-21. (canceled)

22. The method of claim 1, wherein the donor fluorophore is Terbium in a concentration of about 1.75 nM to about 30 nM, and the acceptor fluorophore is BODIPY in a concentration of about 50 nM to about 1 μM, the concentrations being relative to a volume of the assay mixture of 15 μL.

23. The method of claim 22, wherein the concentration of Tb is about 7.5 nM.

24. The method of claim 22, wherein the concentration of Tb is about 15 nM.

25. The method of claim 22, wherein the concentration of BODIPY is about 250 nM.

26. The method of claim 1, wherein the donor fluorophore is Terbium, in a concentration of about 1.75 nM to about 30 nM, the acceptor fluorophore is BODIPY, in a concentration of about 50 nM to about 1 μM, at least one of the first and second reagents is the full-length Spike protein, the concentrations being relative to a volume of the assay mixture of 15 μL.

27. The method of claim 26, wherein concentration of BODIPY is about 250 nM.

28. The method of claim 1, wherein the donor fluorophore is Eu and the acceptor is ALEXA647.

29. The method of claim 1, which detects an anti-acute respiratory syndrome coronavirus 1 (SARS CoV-1) antibody, or which detects an anti-acute respiratory syndrome coronavirus 2 (SARS CoV-2) SARS CoV-2 antibody, or which detects an anti-Middle East Respiratory Syndrome-related coronavirus (MERS-CoV) antibody.

30.-31. (canceled)

32. An assay kit for homogeneous, TR-FRET-based method for detection of an anti-β-CoV antibody in a patient fluid sample, comprising:
a) first and second reagents comprising a first subpopulation of a β-CoV antigen and a second subpopulation of the β-CoV antigen, respectively, wherein the first and second subpopulations are differentially labeled with a donor fluorophore and an acceptor fluorophore, wherein the first and second reagents may be disposed in the same or different containers; or
b) a first reagent comprising a β-CoV antigen and a second reagent comprising at least one secondary antibody or a nanobody that binds the anti-β-CoV antibody, wherein the first and second reagents are differentially labeled with the donor fluorophore and the acceptor fluorophore; and
c) printed instructions for using the reagents in the homogeneous, TR-FRET-based method for detection of the anti-β-CoV antibody in a patient fluid sample.

33. The assay kit of claim 32, wherein the donor fluorophore is a lanthanide metal, or a complex thereof, or wherein the donor fluorophore is a chelate or cryptate of terbium or europium.

34. The assay kit of claim 33, wherein the lanthanide metal is terbium or europium, or wherein the donor fluorophore is terbium.

35.-36. (canceled)

37. The assay kit of claim 32, wherein the acceptor fluorophore is selected from the group consisting of organoboron fluorescent dyes, allophycocyanins, rhodamines, cyanines, squaraines, coumarins, proflavins, acridines, fluoresceins, boron-dipyrromethene derivatives, sodium 6-amino-9-(5-((aminomethyl)carbamoyl)-2-carboxyphenyl)-3-iminio-3H-xanthene-4,5-disulfonate, and nitrobenzoxadiazole, and 2-[5-[3,3-dimethyl-5-sulfo-1-(3-sulfopropyl)indol-1-ium-2-yl]penta-2,4-dienylidene]-3-methyl-3-[5-oxo-5-(6-phosphonooxyhexylamino)pentyl]-1-(3-sulfopropyl)indole-5-sulfonic acid (ALEXA647).

38. The assay kit of claim 37, wherein the acceptor fluorophore is an organoboron dye.

39. The assay kit of claim 38, wherein the organoboron dye is boron-dipyrromethene (BODIPY).

40. The assay kit of claim 32, wherein the β-CoV antigen comprises a β-CoV CoV-2 Spike Protein or an antigenic portion thereof.

41. The assay kit of claim 40, wherein the Spike Protein comprises subunit 1 of Spike protein (S1), subunit 2 of Spike protein (S2), or the receptor binding domain (S1-RBD).

42.-43. (canceled)

44. The assay kit of claim 32, comprising a first set of first and second reagents comprising a first subpopulation of a β-CoV antigen and a second subpopulation of the β-CoV antigen, respectively, wherein the first and second subpopulations are differentially labeled with a donor fluorophore and an acceptor fluorophore, wherein the first and second reagents may be disposed in the same or different containers, and a first further container having disposed therein a different second reagent comprising at least one secondary antibody or a nanobody that binds the anti-β-CoV antibody and that is labeled with the donor fluorophore.

45. The assay kit of claim 44, further comprising a second further container having disposed therein a second reagent comprising the at least one secondary antibody or the nanobody that binds the anti-β-CoV antibody and that is labeled with the acceptor fluorophore.


SEQUENCE LISTING (8 sequences)

SEQ ID NO: 1 - protein, 1356 aa, Human coronavirus
MKLFLILLVLPLASCFFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDSIQADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

SEQ ID NO: 2 - protein, 1273 aa, Human coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1255 aa, Human coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 4 - protein, 1353 aa, Human coronavirus
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPHVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 5 - DNA, 1170 bp, Human coronavirus
atggctacagtcaaatgggctgatgcatctgaaccacaacgtggtcgtcagggtagaataccttattctctttatagccctttgcttgttgatagtgaacaaccttggaaggtgatacctcgtaatttggtacccatcaacaagaaagacaaaaataagcttataggctattggaatgttcaaaaacgtttcagaactagaaagggcaaacgggtggatttgtcacccaagttacatttttattatcttggcacaggaccccataaagctgcaaaatttagagagcgtgttgaaggtgttgtctgggttgctgttgatggtgctaaaactgaacctacaggttacggtgttaggcgcaagaattcagaaccagagataccacacttcaatcaaaagctcccaaatggtgttactgttgttgaagaacctgactcccgtgctccttcccgttctcagtcaaggtctcagagtcgcggtcgtggtgaatccaaatctcaatctcggaatccttcaagtgacagaaaccataacagtcaggatgacatcatgaaggcagtcgctgcggctcttaaatctttaggttttgacaagcctcaggaaaaagacaaaaagtcagcgaaaacgggtactcctaagccttctcgtaatcagagtccttcttcttttcaatctgctgccaagattcttgctcgttctcagagttctgaaacaaaagaacaaaagcatgaaatgcaaaagccacggtggaaaagacagcctaacgatgatgtgacatctaatgtcacacaatgttttggccccagagaccttgaccacaactttggaagtgcaggtgttgtggccaatggtgttaaagctaaaggctatccacaatttgctgagcttgtgccgtctacagctgctatgctttttgatagtcacattgtttccaaagagtcaggcaacactgtggtcttgactttcaccaccagagtgactgtgcccaaagaccatccacacttgggtaagtttcttgaggaattaaatgcattcactagagaaatgcaacaacagcctcttcttaaccctagtgcactagaattcaacccatcccaaacttcacctgcaactgttgaaccagtgcgtgatgaagtttctattgaaactgacataattgatgaagtcaactaa

SEQ ID NO: 6 - protein, 389 aa, Human coronavirus
MATVKWADASEPQRGRQGRIPYSLYSPLLVDSEQPWKVIPRNLVPINKKDKNKLIGYWNVQKRFRTRKGKRVDLSPKLHFYYLGTGPHKAAKFRERVEGVVWVAVDGAKTEPTGYGVRRKNSEPEIPHFNQKLPNGVTVVEEPDSRAPSRSQSRSQSRGRGESKSQSRNPSSDRNHNSQDDIMKAVAAALKSLGFDKPQEKDKKSAKTGTPKPSRNQSPSSFQSAAKILARSQSSETKEQKHEMQKPRWKRQPNDDVTSNVTQCFGPRDLDHNFGSAGVVANGVKAKGYPQFAELVPSTAAMLFDSHIVSKESGNTVVLTFTTRVTVPKDHPHLGKFLEELNAFTREMQQQPLLNPSALEFNPSQTSPATVEPVRDEVSIETDIIDEVN

SEQ ID NO: 7 - protein, 419 aa, Human coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 8 - protein, 362 aa, Human coronavirus
QHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA